(19) United States
(12) Patent Application Publication
(10) Pub. No.: US 2005/0048564 A1
(43) Pub. Date: Mar. 3, 2005

(54) Protein expression profile database

(76) Inventors: Andrew Emili, Ontario (CA); Gerard Cagney, Dublin (IE)

Correspondence Address: Jonathan M Dermott;Synnestvedt & Lechner, 2600 Aramark Tower, 1101 Market Street, Philadelphia, PA, 19107-2950, US

(21) Appl. No.: 10/479,270

(22) PCT Filed: May 30, 2002
(86) PCT No.: PCT/CA02/00801

(30) Foreign Application Priority Data
May 30, 2001 (CA) 23449265

(51) Int. Cl. G01N 33/53

(52) U.S. Cl. 435/7.1; 702/19

(57) ABSTRACT

This invention describes the use of peptide profiling to identify, characterize, and classify biological samples. In complex samples, many thousands of different peptides will be present at varying concentrations. The invention uses liquid chromatography and similar methods to separate peptides, which are then identified and quantified using mass spectrometry. By identification it is meant that the correct sequence of the peptide is established through comparisons with genome sequence databases, since the majority of peptides and proteins are unannotated and have no ascribed name or function. Quantification means an estimate of the absolute or relative abundance of the peptide species using mass spectrometry and related techniques including, but not limited to, pre- or post-experimental stable or unstable isotope incorporation, molecular mass tagging, differential mass tagging, and amino acid analysis.

### CROSS REFERENCE TO RELATED APPLICATION

This application claims priority from Canadian Patent application No. 2,349,265, which is incorporated by reference herein.

### FIELD OF THE INVENTION

The field of this invention relates to the fields of peptide separation and proteomics, bioinformatics, metabolite profiling, medicine, drug screening and computer databases.

### BACKGROUND OF THE INVENTION

Modem biochemistry and molecular medicine is entering the post-genomic era. While genome sequencing has generated a large amount of genetic data, the focus in the biological sciences is now changing to the full characterization of proteins. Protein post-translational modifications, protein localization, protein-protein interactions, and analysis of protein structure and folding have become subjects of major importance.

Proteomics is the study of patterns of protein expression by complex biological systems. It involves, in principle, the determination of the relative abundance, post-translational modification, and/or stability of large numbers of cellular proteins at specific time-points within the life cycle of an organism.

There is growing recognition that qualitative and quantitative analysis of protein expression profiles on a genome-wide scale will accelerate the development of powerful new diagnostic tools and therapeutics, including novel biomarkers and drug targets, as well as lead to a better understanding of the basic molecular logic that governs cell biology. This is because most, if not all, complex biological processes are ultimately regulated by means of protein turnover and not simply through the control of gene expression.

The study of protein expression will bring researchers closer to the actual biological function of genes than studies of gene sequence or gene expression alone. This is because molecular regulation of proteins, and not simply their corresponding genes, holds the key to the function of most, if not all, complex biological processes.

In contrast to genomics, which captures DNA information that is largely stable throughout the lifetime of an organism, proteomics efforts seek to summarize the protein-expression patterns of dynamic biological systems at different times; While there are a finite number of genes in a given genome, a cell's proteome is constantly fluctuating in response to environment and cellular perturbations. Hence, understanding how proteins work together requires systematic data on the entire spectrum of protein status in a cell at any given time.

Biology Enters the Post-Genomic Era

By the late 1990's the DNA sequences of numerous bacterial and eukaryotic organisms had been published and in 2000 the nearly complete DNA sequence of Homo sapiens was completed. The availability of large-scale genomic sequencing efforts now offers investigators a unique opportunity to perform comparative analysis from an evolutionary perspective which can both help to annotate and validate completed genome sequences and also help identify conserved protein function, regulation, or pathways based on protein sequence homology.

Today several disciplines, in particular bioinformatics, functional genomics, and proteomics, are converging in efforts to exploit this newly-available genome sequence information. The long-term objective of these efforts is to understand the function and interrelationships of the many thousands of genes and proteins present in human cells, with the implicit expectation that this understanding will lead to dramatic progress in the clinical sciences.

In the last few years, laboratories have begun to investigate the functions of the protein products of genes and their respective regulatory pathways in a systematic global manner. Several approaches are now commonly used. First, systematic two-hybrid experiments can be used to define interactions among large sets of proteins (Flores et al, 1999), including whole yeast proteome (Ito et al., 2000; Uetz et al, 2000). Second, comprehensive screening of mutant genetic loci as a means for dissecting networks of interacting gene products has recently been adapted to automated high-throughput formats. Finally, powerful experimental tools for identifying the components of protein samples, including large complexes such as the ribosome (Link et al., 1999) and nuclear pore (Rout et al., 2000), and most recently whole organelles and whole cells have been described.

Tandem Mass Spectrometry

Because the amino acid sequence of a protein is encoded in DNA, and because the rules for determining the primary amino acid sequence of a protein are known, vast numbers of hypothetical proteins with no known function await classification and characterization. Clearly, many of these genes and proteins play a role in human disease and other phenomena of biological or commercial interest.

The emerging field of proteomics research relies on enabling technologies that can accurately and rapidly characterize the numerous diverse proteins typically found in biological samples This requires scalable, robust, and automated methods for protein analysis.

To reveal biochemical pathways and regulatory networks, and help define new targets for structure-function analysis, proteomics studies require high-resolution, high-sensitivity techniques for separation, detection, and quantitation of proteins as well as methods for linking proteins to their corresponding cognate gene sequences.

Mass spectrometry (MS) is currently the method of choice for identifying proteins present in biological mixtures. The primary advantages of MS are its high-sensitivity, accuracy and capacity.

Mass spectrometry is the study of gas phase ions as a means to characterize the structures, and hence identities, of molecules. Proteomics began with the commercialization of soft ionization techniques in the 1990s, in particular electrospray ionization (ESI) and matrix assisted laser desorption ionization (MALDI), which permitted analysis of proteins for the first time. Commercial MS instruments are designed as high performance instruments for structural characterization of ions produced by these soft ionization techniques and have largely replaced traditional Edman chemical sequencing for the analysis of proteins. MS has proven to be very successful at identifying limited numbers of proteins, such as single polypeptide bands cut from polyacrylamide gels, and it is currently possible to identify proteins at picomolar to sub picomolar levels.

Recent advances in mass spectrometry and data analysis described below are providing the necessary tools for implementation of high-throughput protein identification and characterization. As the scope of protein analysis has shifted from a molecule-by-molecule approach to a genomic scale, the ability of both academia and industry to generate new MS data has dramatically outstripped the ability to validate, manage, and interrogate the data.

For these studies, routine access to state-of-the-art mass spectrometry instrumentation with an adequate infrastructure is essential. Two new ionization techniques, MALDI and ESI, have revolutionized the analysis of proteins. The MALDI and ESI techniques can be coupled with various types of mass analyzers, such as quadrupoles (Quad, Q), time-of-flight (TOF), ion-trap, Fourier transform ion cyclotron resonance (ICR) and hybrid instruments with two different mass analyzers (Q-TOF). Each kind of instrument has advantages and disadvantages and, in practice, the achievement of high throughput in conjunction with reliable protein identification requires access to both MALDI and ESI instruments.

Mass spectrometry is the most powerful physical technique in its ability to resolve and identify rapidly the thousands of proteins expressed by a genome. Mass spectrometric techniques are particularly effective when coupled with classical biochemical techniques such as proteolytic digestion, immunoprecipitation and separation techniques such as affinity chromatography, HPLC or capillary electrophoresis.

Tandem mass spectrometry (MS/MS) provides a means for fragmenting a mass-selected ion and measuring the mass-to-charge ratio (m/z) of the product ions that are produced during the fragmentation process. The MS/MS process used most often is based on collision-induced dissociation (CID), in which a mass-selected ion is transmitted to a high-pressure region of the instrument where it undergoes low energy collisions with inert gas molecules.

As a molecular ion collides, a portion of its kinetic energy is converted into excess internal energy rendering the ion unstable, and driving unimolecular fragmentation reactions prior to leaving the collision cell. Detailed structural information is generated as a result of fragmentation. The mass selectivity of many commercial MS systems permit the isolation of single precursor peptide ions from mixtures, thereby removing the contribution of any other peptide or contaminant from the sequence analysis step. The product ion spectra can subsequently be interpreted to deduce the amino acid sequence of a protein.

A protein to be identified by MS is first digested enzymatically with a site-specific protease such as trypsin (which cleaves after lysine and arginine residues) in order to produce peptides with structures suitable for MS. Tryptic peptides are particularly amenable to MS/MS analysis since mobile protons localize to the N-terminal amine and the side chains of the carboxy-terminal arginine or lysine residues at which proteolysis occurs. These protons cause peptides to fragment in a somewhat predictable manner following activation in a tandem MS, leading to production of two broad classes of fragment ions—the so-called amino-terminal b-type ions and carboxy-terminal y-type ions. Recognition of the members of these series is a fundamental process of MS-based protein sequence interpretation.

Tandem mass spectrometry is a uniquely powerful technology for identifying the components of low abundance protein complexes (Andersen et al., 1996). Using this technique, the molecular weight of individual ionized peptides resulting from trypsin digestion of protein sample is initially determined by the mass spectrometer. The peptides are then isolated based on their mass/charge properties, fragmented using low energy collision with inert gas (or with resonance excitation), and the fragments are analyzed using a second round of mass spectrometry.

The relative abundance of daughter product ions in peptide tandem mass spectra varies considerably, and some are not observed. This variation reflects subtle differences between favored and disfavored fragmentation sites, the nature of the amino acid side chains, and their position on the peptide backbone. CID of protonated peptides also leads to other fragmentation reaction products that can complicate spectral interpretation. Molecular losses of water or ammonia for instance, are commonly observed in the product ion scans of tryptic peptide ions. Spectra often also contain non-peptide noise peaks. Because of this, de novo interpretation of spectra is extremely difficult to automate and most MS-based identification techniques rely on reducing the computational scale of the problem by searching protein sequence databases using a relatively simple correlation algorithm.

The fragmentation patterns of the peptides can be used to obtain amino acid sequence information by comparison with predicted patterns obtained from translated protein databases. In addition, advances in tandem mass spectrometry mean that polypeptides can now be identified at a low picomolar to femtomolar level in a rapid, sensitive, and versatile manner. By revealing the composition of biologically relevant, low abundance protein complexes, the technology can provide fundamental insight into the circuitry of interacting proteins.

Tryptic peptides are particularly amenable to MS/MS analysis since mobile protons localize to the N-terminal amine and the side chains of the carboxy-terminal arginine or lysine residues at which proteolysis occurs. These protons cause peptides to fragment in a somewhat predictable manner following activation in a tandem MS, leading to production of two broad classes of fragment ions—the so-called amino-terminal b-type ions and carboxy-terminal y-type ions (a typical MS/MS peptide spectra showing prominent b- and y-ions is shown below).

The fragmentation pattern reflects the dissociation of the peptides along the peptide bond backbone, and therefore correlates with the sequence of amino acids for those peptides. Recognition of the members of the b- and y-ion series is a fundamental process of MS-based protein sequence interpretation. Since de novo interpretation of spectra is difficult to automate, most MS-based identification techniques rely on reducing the computational scale of the problem by searching protein sequence databases using a relatively simple correlation algorithm. The SEQUEST program (U.S. Pat. No. 5,538.897), for instance, uses uninterpreted product ion spectra to search databases of theoretical spectra derived from protein and translated gene sequence databases.

Recent developments in tandem mass spectrometry (MS/MS) now allow for the identification of hundreds of proteins per sample in a single run using available technology. This represents a major breakthrough compared to traditional methods, for example, 2D gel electrophoresis, and permits, for the first time, protein analysis on a truly proteomic scale.

Accurate mass measurement of peptides derived from proteins provides information not available from DNA sequence, such as post-translational modifications and correction to errors in the DNA databank. Database searching with masses of peptides obtained from proteolytic digests is a well-established technique in many laboratories around the world. The searching of databases with partial sequence information obtained from MS/MS sequencing experiments is even more reliable because it imposes statistical constraints on the identification.

The ability of mass spectrometry techniques to quantify the levels of individual peptides in a sample has been limiting. Recent approaches, such as ICAT (isotope-coded affinity tags; Gygi et al, 2000), have begun to address this issue. Using ICAT and similar strategies, the proteins of two samples are differentially modified with a reagent that quantitatively adds a molecular tag of defined molecular mass to one of the protein samples. By combining the samples after this treatment, the relative abundance of different protein species in each sample can be estimated by comparing the signal intensities of the corresponding peptides in the mass spectrometer.

Another quantitative approach, limited to culturable organisms, is to label growth media with stable isotopes such as N15. The isotope becomes incorporated into the peptide or protein and the isotope-treated peptide is offset in the mass spectrum by multiples of 1 amu (the difference in mass between the naturally abundant isotope N14 and the heavy isotope derivative N15) depending on the number of N atoms in the peptide. These spectra can be deconvoluted to determine the relative abundance of the labeled and unlabeled peptide species. Alternatively, non-isotopic mass tags, whereby the ‘labeled’ or tagged species is offset by the mass of the tag, can be used. Thus methods suitable for high-throughput and efficient identification and quantitation of large numbers of proteins from complex mixtures are now available.

HPLC

High-resolution separation techniques are required to separate the peptide components of complex biological mixtures prior to mass spectrometry. A particularly powerful approach to identifying the components of complex protein mixtures is direct analysis of the protease-digested proteins using high-performance, high-resolution multi-dimensional liquid separation techniques coupled online to mass spectrometry/database searching (HPLC-MS/MS)(Link et al., 1999). This strategy enables the separation of very complex peptide mixtures, such as the whole cell extracts or nuclear extracts (Washburn, 2000). One aspect of the method separates complex peptide mixtures by strong cation exchange in the first dimension and by reverse phase in the second. However, many combinations of separation media and more than two dimensions could be used. One advantage of the strategy is that it eliminates the need to separate proteins on gels or to identify them using antibody- or affinity-based techniques that are both time-consuming and difficult to standardize. Therefore this technique circumvents the technical and analytical limitations associated with traditional proteomics technologies.

Bioinformatics

The interpretation of peptide mass spectra for the purposes of generating protein identifications can be carried out manually but requires experience and skill and is prohibitively time-consuming. For this reason, computer algorithms have been developed that, while not capable of interpreting all spectra they encounter, can easily outperform human identifications for even minimally complex peptide mixtures. Any of several generally available algorithms may be used for this purpose. For instance, the SEQUEST program (Eng et al., 1994) uses uninterpreted product ion spectra to search databases of theoretical spectra derived from protein and translated gene sequence databases. SEQUEST first generates a list of theoretical peptide masses for each entry in the database that match the experimentally determined peptide mass, producing a list of candidate peptides. The program then calculates the fragment ion masses expected for each of the candidate peptides, generating a predicted MS/MS spectrum. Finally, the experimentally determined MS/MS spectrum is compared with the predicted spectra using a correlation function. Each comparison receives a score, and the highest-scoring peptide(s) are reported. When high scoring matches are detected, one effectively jumps from spectral data directly to a peptide identity, which in turn can be linked to the entire amino acid and DNA sequence of the corresponding gene. Ideally, a protein is positively identified when the spectra of one or more peptides in a tryptic digest can be matched unambiguously.

Mass spectral reference libraries representing stored tandem mass spectra, or validated chemical signatures, are routinely used for the identification of small chemical compounds by MS (eg. Wiley Registry, NIST database). Unknown compounds can then be both identified by searching experimental spectra against a comprehensive database of these reference mass spectra, which are in turn derived from pure compounds, so that only hits of strong similarity or identity are produced. A similar reference spectral database approach would likewise facilitate MS-based identification of proteins.

Compared to mRNA expression analysis the development of corresponding ‘proteomics’ technologies has lagged, with only a few laboratories addressing complex phenotypes on a global scale. Nonetheless, protein expression profiling holds great promise for rapid genome functional analysis. It is plausible that the protein expression profile could serve as a universal and rich cellular phenotype: provided that the cellular response to disruption of different steps of a given biochemical process or pathway is similar, and that there are sufficiently unique cellular responses to the perturbation of most cellular pathways, systematic characterization of novel genetic mutants could be carried out with a single genome-wide protein expression measurement.

To date the only studies focusing on peptides or proteins that includes a quantitative component has been the separation of bacterial and yeast cell lysates on 2-dimensional electrophoretic gels (refs). These approaches do not directly identify the resolved proteins, are relatively insensitive, and are unlikely to scale up to the study of larger proteomes (e.g. that of vertebrates). Furthermore, no attempt was made to use the data to identify or characterize unknown samples.

### SUMMARY OF THE INVENTION

The protein profiling approach proposed has both a qualitative and a quantitative component such that each profile generated can be directly compared to other profiles present in a reference database.

This invention describes the use of peptide profiling to identify, characterize, and classify biological samples. In complex samples, many thousands of different peptides will be present at varying concentrations. The invention uses liquid chromatography and similar methods to separate peptides, which are then identified and quantified using mass spectrometry. By identification it is meant that the correct sequence of the peptide is established through comparisons with genome sequence databases, since the majority of peptides and proteins are unannotated and have no ascribed name or function. Quantification means an estimate of the absolute or relative abundance of the peptide species using mass spectrometry and related techniques including, but not limited to, pre or post-experimental stable or unstable isotope incorporation, molecular mass tagging, differential mass tagging, and amino acid analysis.

The principle experimental strategy of the present invention is centered on rapid high-throughput protein identification using coupled tandem mass spectrometry (MS/MS) and sequence database searching. Quantitation is based on either metabolic labeling with stable isotopes or with chemical derivation. Below, an example of a non-isotopic tag based on the lysine-specific guanidylation reagent O-methylisourea is described in detail. Significant patterns of peptide expression are identified with software and data mining algorithms. Below, a method is described for identifying, classifying and characterizing functions of known and unknown gene products, peptides and proteins, for characterizing metabolic and other functional pathways in cells, and for identifying the proteins and pathways targeted by drugs and other reagents. The method is based on the comparison of protein profiles obtained following global proteomics or other comprehensive protein studies from cells, cell fractions, tissues, organisms or other defined sources.

The invention further contemplates the use of high-throughput robotic screening of diverse chemical compound libraries to systematically identify small molecules that perturb cellular pathways associated with disease. The protein targets of the lead compounds will be isolated and identified by the tandem mass spectrometry profiling techniques described herein. Protein profiling acts as an optimal assay since the profile of a healthy cell or tissue is the goal.

The invention relates to a method for identifying the constituent proteins for a cell type, tissue or pathological sample using a database comprising peptide profile libraries wherein the libraries have multiple peptide sequences, comprising;
1. deriving a plurality of peptides from the cell type, tissue or pathological sample;
2. identifying the peptide species by liquid phase tandem mass spectroscopy sequencing;
3. compiling a data set or peptide profile containing the collection of peptide sequences obtained thereby; and
4. cross-tabulating with a collection of peptide sequences in the database.

The step of deriving a plurality of peptides from the cell type, tissue or pathological sample preferably further comprises the step of:
a) obtaining a peptide-containing extract of the cell type, tissue or pathological sample;
b) digesting the extract producing peptides with an enzyme, the enzyme capable of localizing mobile protons to the N-terminal amine and the side chains of the carboxy-terminal arginine or lysine residues;
c) separating the peptides by high pressure liquid chromatography apparatus,

The enzyme preferably comprises one selected from the group consisting of trypsin and endoproteinase LysC. The step of digesting the extract producing peptides preferably further comprises the steps of:
a) dividing the extract into two equal portions;
b) derivatizing completely one of the two equal portions with a reagent, the reagent comprising one selected from the group consisting of o-methylisourea, homoarginine, canavanine, hydrazine, phenylhydrazine, and butyric acid derivatives.
c) combining the two portions.

The methods of the invention may be used in toxicology analysis. The methods optionally comprise administering a candidate compound to a cell. As described above, samples suitable for MS anaylsis are generated and a peptide profile is produced. Relative abundance of peptides in samples is also preferably determined. This candidate compound peptide profile is compared to peptide profiles in a database or library (for example, profiles showing the cell in a normal state and in varied states of toxicity). If the candidate compound sample profile is highly similar to (for example, greater than 90%, 95%, or 99% similarity), or identical to a profile in the database or library, then that similarity shows the amount of toxicity of the candidate compound to the cell. If the candidate compound sample profile is highly similar to a normal cell profile, than the candidate compound is less likely to be toxic than if the candidate compound sample profile is similar to the peptide profile of the cell in state of toxicity. The relative abundance of the test sample peptides is also preferably compared to other profiles to determine the amount of toxicity of a candidate compound. In a similar manner, candidate drugs compounds may be screened against cells, such as diseased cells. If the candidate drug shifts the profile from a disease profile and relative abundance towards a normal, healthy profile and relative abundance with substantial similarity (eg. Over 90%, 95%, 95% similarity), or identical to the healthy profile and relative abundance, the drug compound is likely to be useful as a therapeutic.

Another embodiment relates to a method for identifying a peptide sequence for a cell type, tissue or pathological sample using a database comprising peptide profile libraries wherein the libraries have multiple peptide sequences, comprising;
a) obtaining a peptide-containing extract of the cell type, tissue or pathological sample;
b) digesting the extract producing peptides with an enzyme capable of localizing mobile protons to the N-terminal amine and the side chains of the carboxy-terminal arginine or lysine residues;
c) separating the peptides by high pressure liquid chromatography apparatus;
d) identifying the peptide species by tandem mass spectroscopy sequencing; and
e) compiling a data set or peptide profile containing the collection of peptide sequences obtained thereby.

The enzyme is preferably selected from the group consisting of trypsin and endoproteinase LysC. The step of digesting the extract producing peptides preferably further comprises the steps of:
a) dividing the extract into two equal portions;
b) derivatizing completely one of the two equal portions with a reagent, the reagent comprising one selected from the group consisting of o-methylisourea, homoarginine, canavanine, hydrazine, phenylhydrazine, and butyric acid derivatives.
c) combining the two portions.

Another aspect of the invention includes a method for quantitating the relative abundance of proteins in two samples of a cell type, tissue or pathological sample using a database comprising peptide profile libraries wherein the libraries have multiple peptide sequences, comprising:
a) deriving a plurality of peptides from each sample of the cell type, tissue or pathological sample,
b) identifying the peptide species by tandem mass spectroscopy sequencing;
c) compiling a data set or peptide profile containing the collection of peptide sequences obtained thereby;
d) cross-tabulating with a collection of peptide sequences in the database of peptide sequences; and
e) determining the relative abundance of the proteins.

In the methods of the invention, a pathological sample may have been contacted with a candidate drug compound and the peptide profile and/or relative abundance of the peptides and/or proteins is compared to a database comprising peptide profile libraries of the cell in varied states of toxicity (ie. exposed to known toxic compounds which injure and/or kill the cell). The toxicity of the candidate drug compound may be determined by comparison of the profile and relative abundance for the cell type, tissue or pathological sample exposed to the candidate drug compound with the profile and relative abundance for the cell type, tissue or pathological sample in varied states of toxicity and a normal state. A similar method may be used to determine whether a compound is likely to be useful as a therapeutic, for example by comparison of the profile and relative abundance for a pathological (diseased) cell type, tissue or sample exposed to the candidate drug compound with the profile and relative abundance for the cell type, tissue or sample in a normal, healthy state.

The invention includes a method for quantitating the relative abundance of proteins in two samples of a cell type, tissue or pathological sample using a database comprising peptide profile libraries wherein the libraries have multiple peptide sequences, comprising:
a) deriving a plurality of peptides from each sample of the cell type, tissue or pathological sample;
b) identifying the peptide species by tandem mass spectroscopy sequencing:
c) compiling a data set or peptide profile containing the collection of peptide sequences obtained thereby;
d) determining the degree of relatedness of a collection of peptide sequences in the database of peptide sequences using clustering and related statistical methods

The step of deriving a plurality of peptides in two samples preferably further comprises the step of:
a) obtaining a peptide-containing extract of each sample;
b) digesting separately the extracts producing peptides with an enzyme, the enzyme capable of localizing mobile protons to the N-terminal amine and the side chains of the carboxy-terminal arginine or lysine residues;
c) combining the two extracts, and
d) separating the peptides by high pressure liquid chromatography.

The enzyme preferably comprises one selected from the group consisting of trypsin and endoproteinase LysC.

The step of digesting the extracts preferably further comprises the step of derivatizing completely one of the two extracts with a reagent, the reagent comprising one selected from the group consisting of o-methylisourea, homoarginine, canavanine, hydrazine, phenylhydrazine, and butyric acid derivatives.

The invention also includes a method for identifying a peptide sequence for a cell type, tissue or pathological sample, comprising:
a) obtaining a peptide-containing extract of a cell type, tissue or pathological sample;
b) digesting the extract producing peptides with an enzyme capable of localizing mobile protons to the N-terminal amine and the side chains of the carboxy-terminal arginine or lysine residues;
c) separating the peptides by high pressure liquid chromatography apparatus:
d) identifying the peptide species by tandem mass spectroscopy sequencing; and
e) compiling a data set or peptide profile containing the collection of peptide sequences obtained thereby.

The enzyme preferably comprises one selected from the group consisting of trypsin and endoproteinase LysC.

The step of digesting the extract producing peptides preferably further comprises the steps of:
a) dividing the extract into two equal portions;
b) derivatizing completely one of the two equal portions with a reagent, the reagent comprising one selected from the group consisting of o-methylisourea, homoarginine, canavanine, hydrazine, phenylhydrazine, and butyric acid derivatives.
c) combining the two portions.

Another embodiment of the invention is a computer system for identifying quantitative peptide profiles, comprising:
(a) a database including peptide profile libraries for a plurality of types of organisms wherein the libraries have multiple peptide profiles each profile comprising an array of at least 50 peptide species each having a unique identifier cross-tabulated with quantitative data indicating relative and/or absolute abundance of each peptide species in a sample; and
(b) a user interface capable of receiving a selection of one or more queries to the database for use in determining a rank-ordered similarity of peptide profiles in the database.

The invention includes a method of producing a computer database comprising a computer and software for storing in computer-retrievable form a collection of peptide profiles for cross-tabulating with data specifying the source of the peptide-containing sample from which each peptide profile was obtained. Optionally, at least one of the sources is from a sample known to be free of pathological disorders. Optionally, at least one of the sources is a known pathological specimen.

The invention also includes a method of comparing quantitative peptide profiles using a database of a plurality of peptide profile libraries, the method comprising:
a) receiving a selection of two or more of the peptide profile libraries;
b) determining the peptide profiles common to the selected peptide profile libraries and identifying profiles unique to each of selected peptide profile library; and
c) displaying the results of the determination.

The correlation of a peptide profile against selected peptide profile libraries may be determined by
P_{x,y}=[1n_{(j=1 to n)}Σ(Xⱼ−μₓ)(Yⱼ−μ_{y})]/[∂ₓ·∂_{y}]
where peptides common to two profiles score ‘1’ and peptides not shared between profiles score ‘0’.

The peptides profiles are preferably of cell fractions, the cell fractions comprising high molecular weight proteins, soluble proteins, membrane proteins, modified proteins, phosphoproteins, peptides terminating in lysine or arginine or the specific products of proteolytic enzymes or chemical derivatives of those products, peptides containing rare amino acids, and proteins isolated by binding to disease-specific affinity reagents.

The specific products of proteolytic enzymes may be comprise chemical derivatives of these products wherein de novo sequencing or relative abundance measurements of the peptides is facilitated.

The chemical derivatives may be obtained by guanidinylation and related modifications. The rare amino acids may comprise tryptophan and cysteine and amino acids comprising 5% or less of the amino acid representation.

The disease-specific affinity reagents may comprise polyclonal antibodies, toxin or drugs. The peptide profiles may be of peptide sequences, the peptide sequences comprising mammalian peptide sequences. Thee peptide profiles may be of peptide sequences, the peptide sequences comprising microbial peptide sequences.

The step of receiving a selection of two or more of the peptide profile libraries for comparison may include receiving a user selection from two or more pull-down menus using a graphical user interface. The step of receiving a selection of two or more of the peptide profile libraries for comparison may comprise command line entry using a computer. The step of receiving a selection of two or more of the peptide profile libraries for comparison may comprise receiving an electronically transmitted file containing sequence and quantitative data. The results of the determination may comprise a unique identifier for related peptide profiles. The results of the determination may comprise annotated information relating to the related peptide profiles obtained from a public database. The results of the determination may comprise quantitative or relative abundance information relating to the related peptide profiles obtained from a public. database. The method may further comprise the step of displaying the peptide profiles common to the selected peptide profile libraries. The method may further comprise the step of displaying the peptide profiles unique to the selected peptide profile libraries.

The invention also includes a method of identifying peptide profiles common to a set of environments, organisms, organs, tissues, cells, cellular fractions or isolated molecular complexes using a database comprising peptide profile libraries for a plurality of types of organisms wherein the libraries have multiple peptide sequences, the method comprising:
(a) displaying at least one list of peptide profile libraries;
(b) receiving a selection of one or more peptide profile libraries from at least one list of peptide profile libraries;
(c) determining peptide profiles common to the selected peptide profile libraries; and
(d) displaying the results of said determination.

### BRIEF DESCRIPTION OF THE DRAWINGS

Embodiments of the invention will be described by way of example and with reference to the drawings in which:

FIG. 1 is a diagram of the MCAT approach for peptide sequencing and relative protein abundance determination.

FIG. 2 is diagram showing how MCAT enables identification and quantitation of complex protein mixtures.

FIGS. 3A and 3B are diagrams showing de novo sequencing of a yeast peptide and a human peptide using MCAT approach.

FIGS. 4A and 4B are diagrams showing relative abundance ratios of positively-identified peptides.

FIG. 5 is a peptide profile generated by a one-dimensional LCMS from diverse human tissues.

FIG. 6 shows proteins identified using MCAT based peptide profiling of seven human tissues.

FIG. 7 shows the differences between protein expression of the seven human tissues highlighted by applying agglomerative clustering algorithms.

FIG. 8 is a similarity dendrogram for different human tissue constructed using peptide profiling.

FIG. 9 is a comparison of peptide profiles of different cell compartments.

FIG. 10 is a comparison of peptide profiles for untreated and leptin-treated human muscle cells.

FIG. 11 shows peptide profiling to distinguish species.

FIG. 12 is a representation of a reference database of protein profiles.

### DETAILED DESCRIPTION OF THE PREFERRED EMBODIMENTS

A Quantitative Peptide Profile serves as a precise fingerprint of peptides that can be successfully isolated, identified and quantified from the myriad of proteins expressed in cells under any given condition. This profile, in turn, can serve as a unique identifier of cell state. This document describes a method to use quantitative peptide profiles to compare biological samples, from any tissue or cell, among different types of cell (e.g. nervous tissue cells), or even in samples where little or no mRNA is made (e.g. blood platelet cells).

The present invention is distinct from the established method of mRNA expression profiling in three important respects.

First, as mentioned above, the relative abundance of an mRNA is not predictive of the abundance of the corresponding protein or cognate peptides. This is because many factors affect protein expression subsequent to the event of mRNA production, including splicing, protein terminal processing, protein localization, protein degradation, protein modification, codon usage, the levels of available amino acids and the subcellular localization of the protein. mRNA expression profiling is unable to account for or predict these events.

Second, the technology used to acquire mRNA and peptide expression data is fundamentally different, the former using nucleic acid hybridization and fluorometric quantitation, with the latter, in this embodiment of the invention, using mass spectrometry and related ionization techniques. The invention includes a method for detecting and quantitatively analyzing peptides in a biological sample, comprising:
a) obtaining a biological sample in a form suitable for coded abundance tagging;
b) identifying and quantitating the peptides in the sample by mass coded abundance tagging.

In one aspect, the method involves:
obtaining an extract of the biological sample, such as a cell extract,
digesting the sample, preferably with an enzyme, such as trypsin, to generate peptides with a terminal amine group, such as a terminal lysine,
contacting the peptides with mass differential reagent, such as a guanidination compound (eg. Lysine guanidination compound, such as o-methylisourea, which modifies the epsilon-amine of the C-terminal lysine),
separating the peptides, preferably with liquid chromatography, such as high throughput capillary liquid chromatography, and
generating mass spectra for the peptides, preferably with electrospray tandem mass spectrometry.

The method is preferably carried out in both orientations, with a sample divided in two and either modified or unmodified. Peptides are alternatively unmodified and modified with o-methylisourea differ by the mass differential encoded by the mass differential reagent (e.g. 42 amu for O-methylisourea). The method preferably further involves sequencing the peptides and/or determining relative abundance of the peptides. Methods of sequencing and determining relative abundance are described below. Sequencing preferably involves comparing pair-wise sets of spectra (MS/MS spectra) to identify identities of y-ion peaks. One can use a short sequence of contiguous amino acid sequence from a peptide (E.g. 5-10 amino acids or greater than 10 amino acids) to identify a corresponding protein.

For identified peptides, the single ion intensity profile is reconstructed from the full scan data and the relative abundance of modified peptides is determined by integrating the area under the curve.

The invention includes a method of identifying a test sample by obtaining a peptide profile for the test sample, preferably by MS. This peptide profile is then compared to peptide profiles in a database or library to determine if the test sample profile is highly similar to (for example grater than 90%, 95% or 99% similarity) to a profile in the database or library. Relative abundance information may similarly be used to identify the test sample.

The methods of the invention may be used in toxicology analysis. The methods optionally comprise administering a candidate compound to a cell. As described above, samples suitable for MS anaylsis are generated and a peptide profile is produced. Relative abundance of peptides in samples is also preferably determined. This candidate compound peptide profile is compared to peptide profiles in a database or library (for example, profiles showing the cell in a normal state and in varied states of toxicity). If the candidate compound sample profile is highly similar to (for example, greater than 90%, 95%, or 99% similarty), or identical to a profile in the database or library, then that similarity shows the amount of toxicity of the candidate compound to the cell. If the candidate compound sample profile is highly similar to a normal cell profile, then the candidate compound is less likely to be toxic than if the candidate compound sample profile is similar to the peptide profile of the cell in state of toxicity. The relative abundance of the test sample peptides is also preferably compared to other profiles to determine the amount of toxicity of a candidate compound. In a similar manner, candidate drugs compounds may be screened against cells, such as diseased cells. If the candidate drug shifts the profile from a disease profile and relative abundance towards a normal, healthy profile and relative abundance with substantial similarity (eg. Over 90%, 95%, 95% similarity), or identical to the healthy profile and relative abundance, the drug compound is very likely to be useful as a therapeutic.

Although mRNA expression profiles from cells treated with different drugs have been compared to each other in order to determine which existing profile most closely matches a ‘novel’ profile (Hughes et al., 2000), this approach has been to date confined to one type of organism, the yeast Saccharomyces cerevisiae.

Using a comprehensive database of reference peptide expression profiles, the pathway(s) perturbed as a consequence of an uncharacterized mutation, pharmaceutical treatment, or developmental or disease state would be ascertained by simply asking which expression patterns in the database the resulting profile most strongly resembles. The database or library will include one or more profiles and/or relative abundance determination and may be electronic or in a hard copy form. A sufficiently large and diverse set of profiles obtained from different mutants, chemical treatments, and environmental conditions would also result in a relatively comprehensive identification of coordinate protein expression sub patterns, allowing hypotheses to be drawn regarding the functions of gene products based on their relationship to other proteins (Elsen et al., 1998).

There are several advantages to this profiling approach compared to the analysis of single peptides or proteins. First, there is no requirement for prior knowledge about the functions of the responsive peptides or parental proteins. Second, protein functions deduced from comparisons of profiles in a database can be derived from very subtle physiological responses. For instance, even though peptide levels may change only slightly in response to an experimental treatment, coordinate changes among many measured peptide abundances can be sufficient to characterize that phenotype. The large numbers of peptides measured make it unlikely that an unrelated physiological state will have an identical profile, even though this may not be apparent when using conventional experiments that measure the levels of one or a few proteins. Third, closely related profiles can be classed together, thus improving our understanding of the underlying biological basis of the classifications.

The invention includes proteins, including drugs, and other compounds identified using methods of the invention.

### EXAMPLES

### Example 1

### Measurement of Protein Relative Abundance in Complex Mixtures

The method relies on modification of peptides at ε-amine of lysine residues with O-methylisourea. Peptides so modified can be readily detected by mass spectrometry because their mass is increased by 42 Da (per lysine residue in the sequence). Therefore, the relative abundance of a single peptide from two different samples can be determined following differential modification with O-methylisourea by comparing the signal intensities for the pair in a mass spectrometer.

The steps of the MCAT procedure are as follows (FIG. 1):
(1) Two protein mixtures, obtained following different experimental treatments of a sample, are digested enzymatically with trypsin.
(2) One digest is treated with O-methylisourea and the other with control buffer.
(3) The digests are desalted using ZipTip reverse phase extraction.
(4) The two mixtures are combined and analyzed by automated electrospray LC-MS/MS. Using either one-dimensional (reverse phase) or two-dimensional (cation exchange and reverse phase) liquid chromatography, the peptides are separated as they are introduced to the mass spectrometer. The instrument is run in automated multistage mode, whereby the following cycle is implemented. First, a full MS scan (400-1600 m/z) is used to record the relative intensities of peptide ions emerging from the column. Next. MS/MS scans of selected ions are used to collect spectra suitable for peptide identification. The instrument then reverts back to full scan mode, but is programmed to exclude MS/MS analysis of ions that have been identified in the previous cycle(s).
(5) The MS/MS spectra are used to identify the peptides using protein database searching algorithms.
(6) For identified peptides, the single ion intensity profile is reconstructed from the full scan data and the relative abundance of modified and unmodified peptides calculated by integrating the area under the curve.

In order to correct for systemic errors, for instance preferential labeling by O-methylisourea of one sample, the experiment is carried out in both orientations, that is both samples are divided in two and either modified or unmodified. The fractions are then combined with the corresponding modified or unmodified fracton from the other sample.

Table 1 shows some top scaring peptides from this analysis and their relative abundance as estimated by the area-under-curve of their respective selected ion tracings. For nearly all peptides, the ratio of unmodified to modified signal is slightly less than the expected 1:1. The variation from ideal 1:1 ratio is not the result of reduced ionization efficiency or MS signal of the modified peptides relative to their unmodified forms because the effect was consistently observed in subsequent experiments independently of which sample was chosen for modification. More likely, it results from preferential recovery of unmodified peptides during the Zip Tip desalting step.

For this reason, when comparing two samples A and B using the MCAT procedure, four mass spectrometry analyses are routinely carried out: I) A versus A^{mod}, II) A versus B^{mod}, III) B versus B^{mod}, and IV) B versus A^{mod}. The ratios of unmodified to modified peptide signals obtained in I and III were used to normalize II and IV respectively, and the combination of III and IV served to independently confirm the quantitative observations.

TABLE 1

Identification and quantitation of peptides
from a yeast whole cell digest.

| | | | | | |
|---|---|---|---|---|---|
| | | | | Observed | Expected |
| Protein | Peptide | Z^{a} | Score^{b} | ratio | ratio |

| | | | | | |
|---|---|---|---|---|---|
| YLR044C | AQYNEIQGWDHLSLLP | 2 | 2.3993 | 1:0.29 | 1:1 |
| | TFGAK | | | | |
| | (SEQ ID NO:1) | | | | |
| YLR044C | TTYVTQRPVYLGLPAN | 2 | 2.6639 | 1:0.2 | 1:1 |
| | LVDLNVPAK | | | | |
| | (SEQ. ID. NO:2) | | | | |
| YLR044C | KLIDLTQFPAFVTPMG | 2 | 3.3881 | 1:0.67 | 1:1 |
| | K (SEQ ID NO:3) | | | | |
| YHR174W | WLTGVELADMYHSLMK | 2 | 4.0552 | 1:0.73 | 1:1 |
| | (SEQ ID NO:4) | | | | |
| YHR174W | GVMNAVNNVNNVIAAA | 2 | 3.2283 | 1:0.48 | 1:1 |
| | FVK | | | | |
| | (SEQ ID NO:5) | | | | |
| YBR118W | TLLEAIDAIEQPSRPT | 3 | 3.3888 | 1:0.63 | 1:1 |
| | DKPLRLPLQDVYK | | | | |
| | (SEQ ID NO:6) | | | | |
| YBR118W | VETGVIKPGMVVTFAP | 2 | 2.5458 | 1:0.23 | 1:1 |
| | AGVTTEVK | | | | |
| | (SEQ ID NO:7) | | | | |
| YEL034W | VHLVAIDIFTGK | 1 | 3.0798 | 1:0.15 | 1:1 |
| | (SEQ ID NO:8) | | | | |
| YKL060C | SPIILQTSNGGAAYFA | 2 | 3.6709 | 1:0.73 | 1:1 |
| | GK (SEQ ID NO:9) | | | | |
| YCR012W | ALENPTRPFLAILGGA | 2 | 2.7650 | 1:0.33 | 1:1 |
| | K (SEQ ID NO:10) | | | | |
| YDR441C | GFVPIRRVGKLPGEC* | 2 | 1.1770 | 1:1.07* | 1:1 |
| | (SEQ ID NO:11) | | | | |
| YGR192C | VINDAFGIEEGLMTTV | 2 | 3.1456 | 1:0.31 | 1:1 |
| | HSLTATQK | | | | |
| | (SEQ ID NO:12) | | | | |

^{a}Peptide charge
^{b}SEQUEST Cross-correlation score

Next, mixtures derived from yeast whole cell extracts containing varying proportions of MCAT-treated and MCAT-untreated sample were analyzed (FIG. 2).

Relative abundance signal from five peptides with high SEQUEST scores showed linearity across two orders of magnitude (FIG. 2). Beyond this range, the weaker signal of the two abundances is indistinguishable from background noise.

Table 2 shows variation in the measured relative abundance for two peptides from the same parent protein (and therefore are present in equimolar concentrations) in three replicate experiments. Experiment-to-experiment variation for these peptides is within 25% and variation within a single experiment for peptides derived from the same protein is within 20% (Table 2).

TABLE 2

Identification and quantitation of two
peptides derived from YLR044C in
three replicate experiments (A, B, C).

| | | Ratio | Ratio | Ratio |
|---|---|---|---|---|
| Protein | Peptide | A:A | A:B | A:C |
| YLR044C | KLIDLTQFPAFVT | 1.00:1.00 | 1.00:078 | 1.00:0.87 |
| | PMGK | | | |
| | (SEQ ID | | | |
| | NO:13) | | | |
| YLR044C | AQYNEIQGWDHLS | 1.00:1.00 | 1.00:0.79 | 1.00:1.03 |
| | LLPTFGAK | | | |
| | (SEQ ID | | | |
| | NO:14) | | | |

Ratio of unmodified to modified peptides (normalized to A:A)

This invention also includes computer systems including software and hardware to implement the above methods. Such systems include a database with the peptide profiles.

### Example 2

### De Novo Peptide Sequencing and Quantitative Profiling of Complex Protein Mixtures Using Mass Coded Abundance Tagging

Introduction

There is growing recognition that qualitative and quantitative analysis of proteins on a genome-wide scale will accelerate the development of powerful new diagnostic tools and therapeutics, and lead to a better understanding of the molecular logic that governs cell behavior. This is because regulation of protein abundance holds the key to the proper function of most biological processes (Pandey & Mann, 2000). Proteomics studies depend on scalable robust, and automated methods for protein identification and quantitation that can routinely characterize the numerous diverse proteins typically found in biological samples.

Mass spectrometry (MS) is currently the technology of choice for identifying proteins present in biological mixtures. The primary advantages of MS are its high sensitivity, accuracy and capacity. Tandem mass spectrometry (MS/MS) provides a means for fragmenting mass-selected precursor peptide ions and measuring the mass-to-charge ratio (m/z) of any product daughter ions produced (Andersen et al., 1996). The process usually produces two principle classes of fragment ions, the so-called N-terminal b-type ions and C-terminal y-type ions. informative high quality MS/MS spectra of tryptic peptides typically show prominent b- and y-ion series. Tryptic peptides are particularly amenable to MS/MS analysis since mobile protons that stimulate the fragmentation process readily associate with the side chains of the C-terminal arginine or lysine residues at which proteolysis occurred

If accurate sequence information is available, computer database search algorithms can rapidly and accurately identify proteins analyzed by MS/MS (Eng et al., 1994; Mann & Wilm, 1994; Taylor & Johnson, 1997, Qin et al., 1997), in effect linking the spectra to a corresponding cognate protein or DNA sequence. When combined with recent developments in tandem mass spectrometry, this approach allows for routine identification of dozens to hundreds of proteins in a single analysis. However, because the possibility of alternative splicing, mutation, and/or post-translational modification is likely to be a significant feature of the proteomes of higher organisms, a facile peptide sequencing method that is independent of sequence databases is desirable.

Manual interpretation of peptide MS/MS spectra for the purposes of protein identification (a process usually referred to as de novo sequencing) is often prohibitively challenging. Factors such as variation in favored fragmentation sites, the effects of the chemical nature of the amino acid side chains and their relative order in a peptide backbone, and the presence of side-products such as neutral loss ions and non-peptide noise peaks. To address this issue, Mann and coworkers pioneered a post-experiment stable isotope labeling strategy whereby the C-termini of tryptic peptides are labeled with deuterated water in order to reduce spectral complexity. Comparison of the modified and unmodified peptide MS/MS product ion spectra allows the C-terminal y-ions to be readily distinguished and, hence, the peptide sequence discerned. The impact of this approach has been restricted, however, by the prohibitive cost of the stable isotope and the high mass resolution required to distinguish the labeled products.

Functional genomics studies using DNA microarray technologies have been used successfully to compare the abundance of thousands of mRNA species from distinct cell states. In contrast, only limited analogous quantitative data has been obtained for protein abundance. As the scope of protein analysis has shifted from a molecule-by-molecule approach to a genomic scale, the ability to generate quantitative protein data has lagged considerably. Chait and coworkers reported the potential of stable N¹⁵ isotope labeling of proteins as a means to determine the relative abundance of select subsets of proteins isolated from cultured yeast cells (Oda et al., 1999). As the isotope becomes incorporated, the mass of the protein becomes offset in a mass spectrum by multiples of 1 amu (the difference in mass between the naturally abundant N¹⁴ isotope and the heavy N¹⁵ isotope derivative) depending on the number of labeled N atoms. Although powerful, this approach is restricted to organisms that can be grown in defined media.

Aebersold and coworkers recently introduced an alternative protein quantitation strategy based on post-experiment stable isotope labeling (Gygi et al, 1999). The ICAT (isotope-coded affinity tag) chemistry uses isotopic variants of a biotin-containing moiety to differentially label cysteine-containing peptide as a means to obtain relative abundance data for proteins found in two distinct samples in a single analysis. Other approaches based on differential stable isotope labeling have been devised (Munchbach et al., 2000). The ICAT method is unique in that it specifically enriches for peptides containing the relatively rare amino acid cysteine, thereby simplifying complex protein mixtures for subsequent MS analysis. The relative abundance of proteins can then be determined by monitoring the ratios of pairwise sets of selected peptide species which are offset by 8 amu. While representing a major advance, the ICAT approach is based on a sophisticated proprietary chemistry that analyzes relatively rare cysteine-containing peptides.

Here, a complementary protein identification and quantitation strategy is described, which is termed Mass Coded Abundance Tagging (MCAT), based on the differential post-experiment labeling of tryptic peptides with the lysine guanidation agent O-methylisourea followed by high throughput capillary liquid chromatography electrospray tandem mass spectrometry (LC-MS/MS). MCAT permits facile de novo sequencing of proteins present at pico- to femtomole levels in complex biological mixtures and provides for robust determination of the relative abundance of proteins in various cell states in a systematic, reproducible and straightforward manner. The development and applications of a systematic protein expression profiling strategy based on the MCAT approach outlined here should serve as a powerful means for characterizing the physiological, development or disease state of cells or organisms at the proteome level.

Results

De Novo Peptide Sequencing Using MCAT

The MCAT sequencing method relies on the selective and quantitative (ie. complete) modification of the ε-amine of C-terminal lysine residues of tryptic peptides with O-methylisourea (FIG. 1A). This reagent specifically end efficiently transforms lysine into homoarginine but does not react with the peptide amino terminus or other side groups (Kimmel, 1967). Peptide derivatization with O-methylisourea has previously been shown to facilitate peptide sequencing by MALDI post-source decay (Hale et al., 2000; Beardsley et al., 2000). Here, it is shown that it can be used to sequence multiple individual peptides from complex mixtures in a single high-throughput electrospray LC-MS/MS analysis.

The MCAT de novo sequencing approach is based on two principles. First, a short sequence of contiguous amino acid sequence from a peptide (5-10 residues) usually contains sufficient information to identify a corresponding unique protein. Second, peptides alternatively unmodified and modified with O-methylisourea differ by the mass differential encoded by the MCAT reagent (42 amu). This allows the identities of the informative y-ion peaks to be readily delineated by comparing pair-wise sets of MS/MS spectra, allowing for systematic sequence determination. The MCAT labeling procedure is simple, economic and easy to perform with complex protein mixtures.

The steps of the MCAT peptide sequencing procedure are as follows: (1) A protein mixture, which can be a purified polypeptide or protein complex, a cell fraction, or a crude cell extract, is first digested enzymatically with trypsin; (2) Half of the digest is derivatized to completion following incubation with an excess O-methylisourea; (3) The digests are desalted by C18 solid phase extraction and combined; (4) The pooled peptide mixture is fractionated by reverse phase HPLC and analyzed by automated ESI MS/MS. The mass spectrometer is operated in an automated dual mode whereby successive scans alternatively record a) the m/z of modified/unmodified peptide pairs as they elute from the column and b) the MS/MS fragmentation pattern of each peptide that has undergone collision-induced dissociation (CID); (5) Following MS analysis, the data are processed to obtain the amino acid sequence identities of the components of the protein mixture. The process is illustrated schematically in FIG. 1B.

Inspection of pair-wise peptide spectra indicates that most ion peaks, notably the b-ion and y-ion series, are retained upon modification (Table 1). Since the C-terminal lysines of completely-processed tryptic digests are specifically labeled, the C-terminal y-ions produced during the MS/MS fragmentatior reaction are mass shifted by the addition of the MOAT moiety. The y-ion peaks of the MCAT-modified peptides are offset by 42 amu (FIG. 2), or by factors of 42 resulting from the addition of a second or a third charge (ie. 21, 14 amu). In contrast, the recorded m/z values for b-ions and chemical noise remain unchanged. Therefore, comparison of MS/MS spectra for each unmodified/modified peptide pair allows ready determination of the y-ion peaks. With high quality spectra, discrimination of a well-defined and continuous y-ions series allows the amino acid sequence of a peptide to be readily deduced. This simplifies the spectral interpretation process, allowing for systematic sequence determination by assigning amino acid masses that correspond to y-ion peak distances using a reference table of monoisotopic amino acid masses. If required, a delta mass corresponding to a possible post-translational modification (e.g. +80.0 amu for phosphorylation on serine, threonine or tyrosine residues) or neutral loss (eg. water or ammonia) can be incorporated into this table.

In a systematic series of studies using a crude yeast cell extract (Table 1), it is established that MCAT provides an effective method for sequencing multiple peptides analyzed by LC-MS/MS. First, the ionization, charge and fragmentation properties of peptides were not greatly affected by the chemical derivatization procedure. Peptides generally have one of three different charge states (+1, +2, or +3), each of which results in a unique spectrum for the same peptide. The spectra of numerous unmodified and modified peptide forms showed similar information content and could be correctly interpreted using database search algorithms with similar efficiency. Second, the modification of lysine-containing peptides occurred in a robust unbiased and reproducible manner. Third, the mass tag (42 amu) added to the treated peptides was easily resolvable by MS regardless of charge state and did not overlap with other common adducts or peptide modifications. Even for a charge state of +3, the delta mass is 14 units, well within the resolution of a mass spectrometer. Fifth, the process simplified the spectral interpretation process so that the area of combinatorial sequence space to be searched was easily within the limits of modern computing technology.

High confidence amino acid sequence was readily obtained for ten peptide spectra using the MCAT approach (Table 1). Good quality spectra were chosen from MS runs analyzing complex protein mixtures from various sources (a bacterial cell lysate, a yeast cell lysate, and a human nuclear extract). Two representative analyses are shown in FIG. 2. The identifications were confirmed using a computer database search algorithm. The SEQUEST algorithm (and similar algorithms) can detect MCAT modified lysine residues unequivocally because modification of a C-terminal lysine following trypsin digestion alters the m/z of y-series ions but not b-series ions relative to the unmodified peptide.

Although carried out manually here, the MCAT sequencing process may be formalized to facilitate automation. First, the mass of the tag (or a factor of it resulting from multiple charges) is added to each peak observed in the unmodified spectrum (above some threshold). The spectrum of the modified peptide is searched for peaks corresponding to these ‘mass-tagged’ peaks, any such peaks being candidate y-ions. Peaks appearing in both spectra are likely to represent b-ions or other ion products and are excluded from the initial analysis. Next, the mass differences between all candidate y-ions are calculated. Mass differences matching the known masses of single or double amino acids are noted and attempts are made to extend the sequence from this starting point in both directions (i.e. higher and lower m/z) using known single or double amino acid masses. The putative sequences can be ranked using a score incorporating factors such as unbroken peak series and correlation of observed peaks with theoretical peaks. Moreover, for each putative y-ion series, the remaining peaks (i. those conserved in the unmodified and modified spectra) are candidate b-ions and therefore can be used to impose further statistical limits on the y-ion designations. In other words, for any identified y-ion sequence ACDEFG, the corresponding sequence GFEDCA should be observed, and the extent of the presence or absence of the corresponding peaks can be factored into the overall score.

Our results are typical of peptide MS/MS experiments in that incomplete y-ion series were generally observed. For high mass y-ions (yn, yn-1), this may occur because of charge repulsion; for low mass y-ions (y2, y3), because ion trap instruments generally fail to resolve ions lower than ˜⅓ the m/z of the precursor ion. Nonetheless, for most peptides examined, up to 8 to 15 continuous y-ions were detected, covering the bulk of the predicted amino acid sequence (Table 1). A properly ordered stretch of 6-7 amino acids is usually sufficiently informative to identify a corresponding protein using the BLAST algorithm.

Table 2 shows that MCAT reagent selectively modifies all lysine-terminated tryptic peptides present in the mixture in a quantitative and robust manner. In order to show that modification by the MCAT reagent is specific and that peptides so modified are recognizable by spectral identification algorithms, LC-MS/MS on a control yeast extract and a yeast lysate that had been treated with O-methylisourea was performed. The acquired MS/MS spectra were typically of high quality, with distinct b-series ion patterns the same for modified and unmodified spectra and the y-series offset by 42 Da, confirming that a C-terminal lysine had been modified (FIG. 2). Moreover, the SEQUEST scores for both modified and unmodified peptides were comparable and typical of high fidelity identifications. Importantly, in no case was an unmodified peptide detected in the treated sample (i.e. yielding high SEQUEST scores). The corollary was also true, with no peptides being significantly scored as being modified in an untreated sample (Table 2).

Comprehensive LC-MS/MS analysis of an untreated and an O-methylisourea modified yeast cell lysate yielded significant SEQUEST scores for 291 peptides. For peptides treated with O-methylisourea, the rate of modification of non-lysine residues, such as arginine or alanine, by O-methylisourea was negligible (data not shown), as reported by others (Kimmel, 1967; Hale et al., 2000; Beardsley et al., 2000). Greater than 95% of SEQUEST-validated peptides containing lysine residues were classified as modified at lysine. In contrast, less than 3% of untreated peptides were scored as modified by SEQUEST, the same rate of false-positive scoring observed for arginine-containing peptides. These false-positives may result from poor quality spectra, or from acetylation or trimethylation of amino acids that generate a gain in mass (monoisotopic) of 42.0106 Da or 42.0471 Da respectively. Such false positives can be easily eliminated upon inspection of MS/MS spectra because the y-ions series do not show the characteristic 42 amu shift.

Limitations to the MCAT sequencing method include the need for good quality spectra exhibiting a near continuous y-ion series. Furthermore, as with all de novo sequence efforts, some ambiguity remains due to the isobaric or near-isobaric nature of certain amino acids (e.g. leucine and isoluecine). The MCAT approach is limited to peptides that terminate with a lysine residue. Tryptic fragments ending with arginine resdues are not modified and, therefore, cannot be sequenced by this approach. If necessary, endoproteinase LysC can be used instead of trypsin to generate peptides ending exclusively in lysine residues (apart from peptides derived from the C-terminus). Finally, it should be noted that incomplete trypsin or LysC digestion can potentially complicate the MCAT sequencing process by causing a mass shift in a subset of b-ions. However, the presence of modified internal lysine residues can be readily detected a priori by searching for parent ion mass shifts of multiples of 42 amu (adjusted for the charge on the ion).

Relative Protein Abundance Determination Using MCAT

The MCAT approach allows the relative abundance of proteins to be compared in two different samples following differential modification of peptides from one of the samples with O-methylisourea. By combining the peptides after treatment, the relative abundance of different protein species present in each sample can be estimated by measuring the signal intensities of the peptide pairs in a full scan MS analysis. The basic MCAT approach for measuring protein abundance is outlined in FIG. 1C.

In general, a first test sample and a second test sample may be an experimental sample (e.g. a sample exposed to a test compound of interest) and a control sample (not exposed to the test compound), respectively. Both samples are preferably enzymatically digested, for example in trypsin, and then one of the samples is treated (derivated) with a reagent to create a mass differential. This reagent may be called a mass differential reagent and is preferably a lysine guanidination compound. It may be, for example, o-methylisourea or any compound suitable for MCAT, that creates amino acids terminating in lysine or a homoarginine ending group or variant (memetic) thereof. The peptide of each test sample are then separated, for example ligand chromatography such HPLC, and subjected to MS. The MS spectra is obtained and the peptides in the first and second samples are identified, for example, by protein database searching. Optionally, the relative abundance of the peptides in the first sample and the second sample are determined, for example, by integrating the area under the curve in a single ion intensity profile. Preferably, the peptide profile and relative abundance in the first and second sample is carried out in both orientations.

MCAT protein quantitation is based on two principles; First, pairs of peptides alternatively unmodified and modified with O-methylisourea can be discriminated during a single MS run, thereby serving as mutual internal references for accurate relative quantitation. In MS, the ratios between the recorded signal intensities of the lower and upper mass components of these ion pairs provide a direct measure of the relative abundance of the two forms of a peptide and, by inference, the corresponding proteins in the original cell pools. Second, the identity of the peptides can be obtained by performing MS/MS during the same analysis.

The steps of the MCAT peptide quantitation procedure are as follows: (1) Two protein mixtures to be compared are obtained following different experimental treatment of a cell or tissue and are digested enzymatically with trypsin; (2) One digest is derivatized with O-methylisourea; (3) The peptides are desalted by C18 solid phase extraction, combined, and the isolated peptides are separated and analyzed by automated multistage LC-MS/MS. The mass spectrometer is operated in a dual mode where two alternative scans cycle repeatedly. First, a full MS scan monitors the signal intensity of peptides eluting from the capillary column. Second, peptide sequence information is generated by selecting peptide ions for CID fragmentation in MS/MS mode. Sequence identification can be done using the de novo approach described above or using a protein database search algorithm. (4) Peptides are quantified by comparing the relative signal intensities of pairs of peptide ions with identical sequence that differ in mass due to lysine guanidination. In practice, an ion intensity profile is reconstructed for each sequenced peptide using the MS data and the relative abundance of modified and unmodified peptides calculated by integrating the area under the curve. The combination of MS and MS/MS data therefore determines the relative quantities and identities of the components of protein mixtures in a single analysis. The approach is illustrated schematically in FIG. 1C.

The MCAT approach serves as an effective method for determining relative abundance of proteins by LC-MS/MS since: (1) O-methylisourea derivatizes all lysine-containing peptides present in the mixture in a quantitative manner; (2) the agent adds a mass tag to the treated peptide that is easily resolvable by the mass spectrometer and that does not overlap with common adduct or peptide modifications; (3) the modification preserves the charge and ionization properties of peptides such that the efficiency of ionization and signal intensity are equivalent; and (4) the modified peptides generally co-elute during standard reverse phase chromatographic separation.

To illustrate the process, the relative abundance determination of the peptide LPWFDGMLEADEAYFK (SEQ ID NO:15) from two replicate yeast whole cell extract experiments is shown in FIG. 3. Base peak chromatograms show many peptides eluting over a 60 min run, while selected ion tracings for the predicted doubly-charged unmodified and modified forms of the peptide show both eluting at 35-36 min (FIG. 3A). A single full scan of an ion trap mass spectrometer operated in MS mode is shown in FIG. 3B. Two prominent ion species are discernable and indicated with respective m/z values 21 m/z units apart (FIG. 36). The fact that the ions co-elute, have a detected mass difference of 21 m/z units, and have identical sequences (data not shown) identifies them as a pair of doubly charged sister peptides. Over the course of the 60 minute elution gradient, more than 2,000 MS scans were automatically acquired. FIG. 3C shows reconstructed ion chromatograms for each of the peptide species. The relative quantities were determined by integrating the curves contouring the respective eluting peaks. The ratio (unmodified modified) was determined as 0 88 (Table 2). The peaks in the reconstructed ion chromatograms appear serrated because the MS system alternates between MS and MS/MS modes in order to both measure ion intensity as well as generate a mass spectrum of selected peptide ions for the purpose of protein identification.

Table 2 shows some representative high-scoring peptides from a representative MCAT LC-MS/MS analysis of a yeast cell extract. In these experiments a 1:1 mixture of unmodified;modified peptides was analyzed, and single ion tracings for select peptides throughout an entire chromatographic run typically showed isolated peaks with the unmodified form co-eluting, or eluting slightly earlier, than the modified form (FIGS. 3A and C). For nearly all peptides examined the ratio of unmodified to modified signal was close to the expected 1:1. The range of signal intensities were generally within two-fold of the unmodified form and the percentage error (the difference between the observed and expected abundances) ranged from 1 to 62% (Table 2). Some exceptions were evident and excluded from the analysis. These included peptides that could be positively identified but whose signal is very weak and peptides containing arginines that were modified in addition to lysine at low frequency. Another category of ion found unsuitable for quantitation were singly-charged ions. It is unclear why this is the case but the signal from singly-charged ions is typically lower than that for doubly- or triply-charged ions, possibly rendering them less likely surpass the intensity threshold required for accurate quantitation.

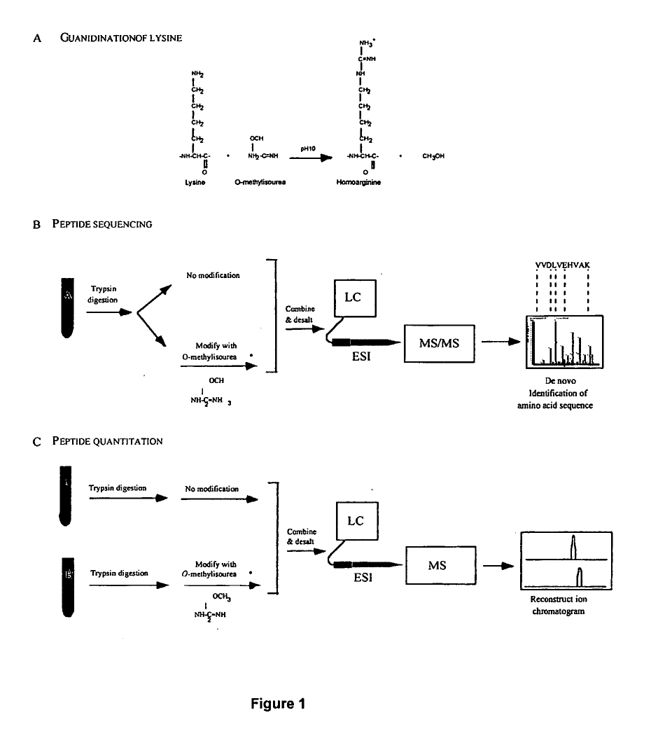

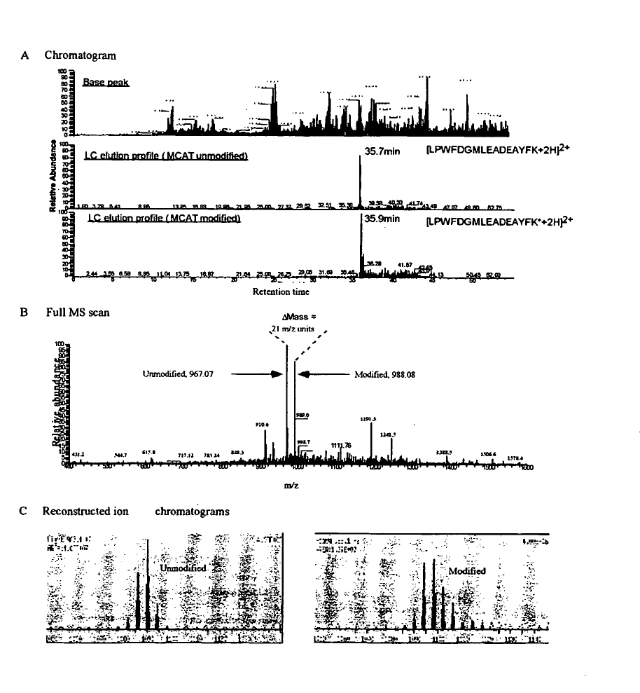

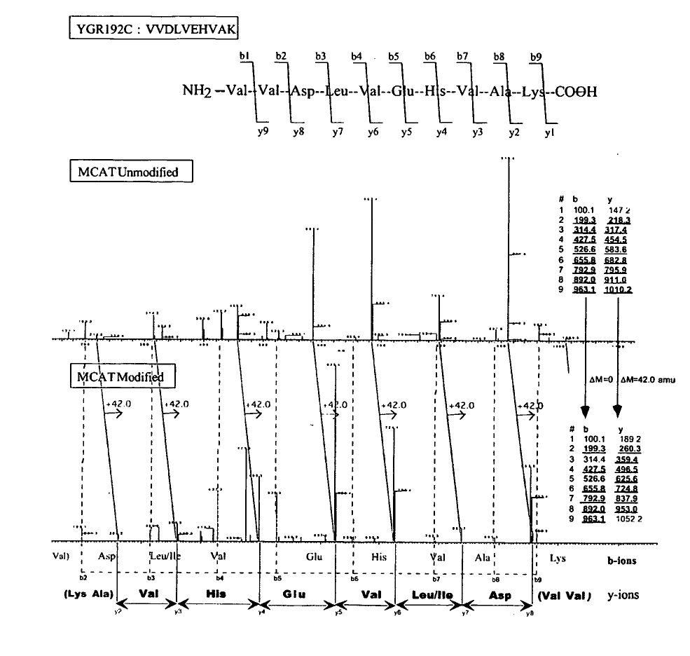

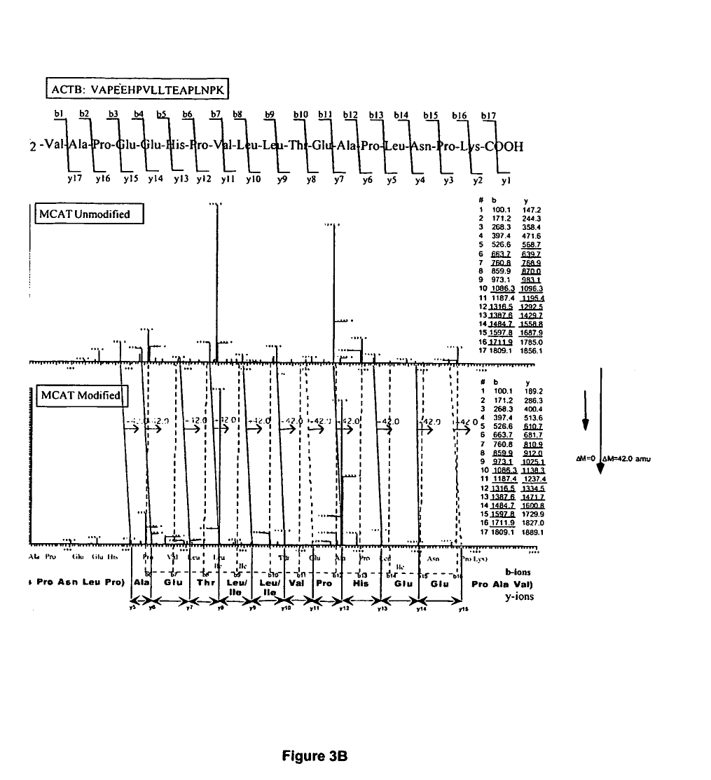

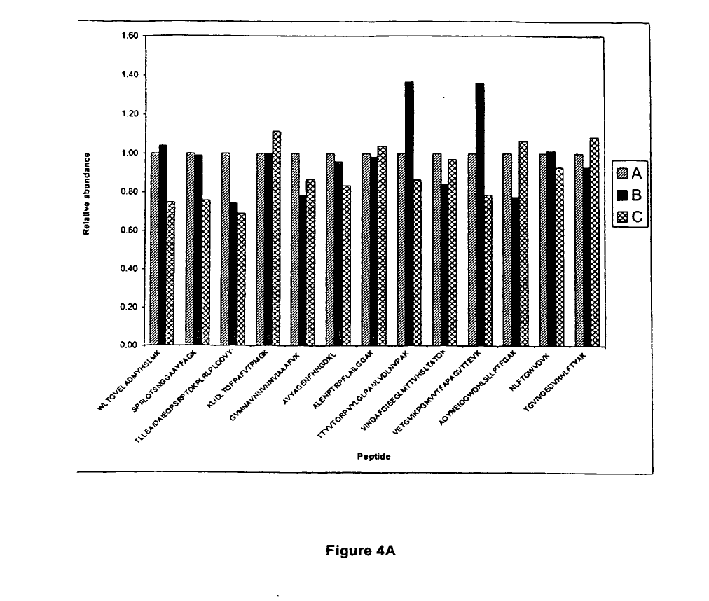

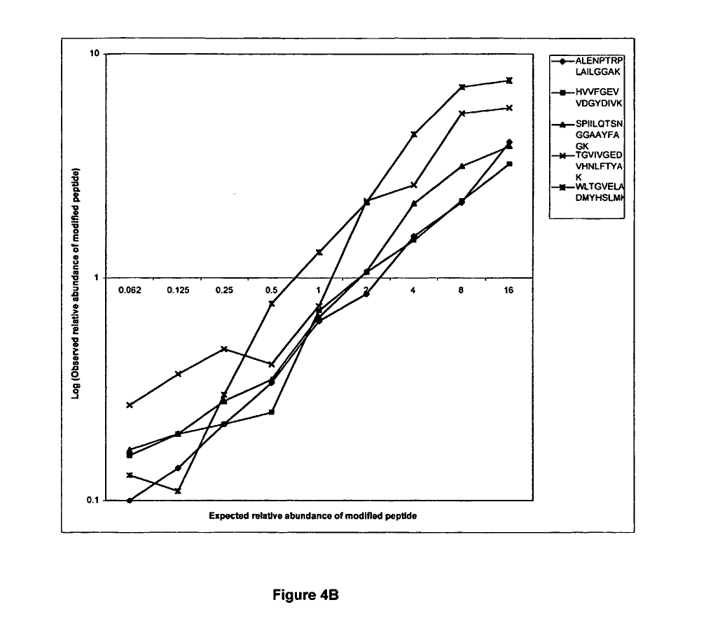

FIG. 4 shows variation in the measured relative abundance for two peptides from the same parent protein (and therefore are present in equimolar concentrations) in three replicate experiments. Importantly, multiple peptides independently analyzed for several proteins gave similar linear responses. Experiment-to-experiment variation for these peptides is within 25% and variation within a single experiment for peptides derived from the same protein is within 20%. The variation from ideal 1:1 ratio is not the result of reduced ionization efficiency or MS signal of the modified peptides relative to their unmodified forms because the effect was consistently observed in subsequent experiments independently of which sample was chosen for modification. More likely, it results from modest variations in peptide recovery during sample workup.

In order to correct for any possible systemic labeling errors, for instance preferential labeling by O-methylisourea of one sample, MCAT quantitation can be carried out in reciprocal orientations. For this reason, when comparing two independent protein samples (A and B), derived for instance from two distinct cell states, the basic MCAT procedure can be carried out in four complementary and reciprocal mass spectrometry analyses: i) unmodified sample A versus modified sample B; II) unmodified sample B versus modified sample A; III) unmodified sample A versus modified sample A; IV) unmodified sample B versus modified sample B. The ratios of unmodified to modified peptide signals obtained in experiments III and IV can be used to systematically normalize and control for variations in the data obtained in experiments I and II, respectively. In practice, the MCAT analysis can be simplified into a two-tiered reciprocal experiment set, I and II, which should independently confirm any significant quantitative observations obtained in a sample comparison.

To confirm the quantitative nature of the MCAT approach, mixtures of modified and unmodified peptides derived from a common crude yeast cell (extract were prepared at various ratios and analyzed by a 30 minute LC-MS/MS analysis. The MS/MS spectra acquired were used to search a non-redundant genome database using the SEQUEST algorithm (Eng et al., 1994) to identify the proteins present in mixtures. The relative ratios of 5 peptide sister pairs was quantified as described above (FIG. 4B). This analysis shows the relative abundance of proteins can be accurately determined (i.e. exhibits a linear response) over a >30 fold dilution series. Beyond this range, the weaker signal of the two abundances was indistinguishable from background noise in these experiments.

It should be emphasized that the data were acquired for polypeptides present at a pico- to femtomole level in a highly complex protein mixture. The loading capacity of capillary reverse phase columns for complex peptide mixtures imposes a strict limit on the detection of low abundance proteins by LC-MS/MS. With a purified protein, most current MS systems generally exhibit a practical dynamic range of roughly three orders of magnitude based on maximal signal to noise ratios that can be acquired (using a purified or low complexity protein preparation). However, sophisticated chromatographic separation techniques can be coupled to fractionate complex peptide mixtures prior to MS in order to substantially improve the detection limits of MS protein analysis (Link et al., 1999; Washburn et al., 2001). Hence, when combined with the MCAT approach, determination of the relative abundance of moderate to low abundance proteins should be achievable even in the absence of enrichment.

An experimental approach for systematically sequencing and quantifying proteins isolated from complex biological mixtures using basic chemistry and mass spectrometry techniques is described and validated. De novo sequencing expands the range of organisms that can be analyzed and removes the reliance on DNA sequence databases that may be incomplete, erroneous, or that fail to account for complexities introduced by alternative splicing, protein modifications, or protein polymorphism. The quantitative capabilities of the method also overcome a significant limitation of current proteomics technologies, whereby the determination of protein abundance on a large-scale is generally low throughput, expensive, and tedious, for instance, radiolabelling of proteins before analysis by two-dimensional gel electrophoresis and quantitation following isolation of individual spots (that may contain one or more polypeptides).

The ICAT method reported by Aebersold and coworkers (Gygi et al., 1999) may significantly improve throughput and reduce sample complexity by enriching for proteins containing the underrepresented amino acid cysteine. These features are useful for sampling a mixture whose proteome complexity could overwhelm the ability of current LC-MS technology to resolve it. The MCAT strategy described here is not limited to any particular affinity chemistry and in principle can be coupled to analogous affinity-based enrichment steps. For this reason, MCAT can potentially be used to identify and quantify all the proteins present in a biological sample. In combination with powerful multi-dimensional LC protein separation techniques, such as that described by Yates and coworkers (Link et al., 1999; Washburn et al., 2001), considerable depth in proteome coverage may be achieved. Quantitative data describing patterns of peptide or protein expression for many hundreds or thousands of proteins can be used to identify or classify protein ‘profiles’ in a similar manner to that routinely used for gene expression data. The combined MCAT approach can therefore be used for identifying, classifying and characterizing functions of known and unknown gene products, for characterizing metabolic and other functional protein pathways in cells, and for identifying proteins and pathways targeted by drugs and other reagents.

The MCAT method offers key experimental advantages.

First, the approach is simple and effective. It builds on established MS techniques and principles that are flexible and can easily be adjusted for large-scale projects, including efforts to generate peptide or protein profiles describing the effects of environment, mutation, disease or experimental interventions such as drug treatment. Significant patterns of expression can be identified with appropriate software and data mining algorithms.

Variations of the MCAT approach can easily be devised, including strategies to address other quantitative aspects of protein expression, those searching for post-translational modifications, or those screening for mutant proteins. It is likely that the number of unique peptide species per organism will be multiplied significantly by the presence of post-translational modifications compared to genome predictions. Because the mass of many common important modifying groups are known, and because their preferences for particular amino acids are often known, the database can be searched for ions predicted to result from peptides with specific modifications.

Finally, the addition of a dynamic component to the molecular descriptions of protein activities is likely to prove critical to our understanding of the biochemical circuitry within cells. Consequently, the development of robust analytical methods, such as the MCAT approach described here, that allow for efficient identification and quantitation of large numbers of proteins from complex mixtures can be expected to have a major impact.

Experimental Protocols

Materials. Media, standard-grade and HPLC-grade laboratory chemicals were obtained from Fischer Scientific (Fair Lawn, N.J.). O-methylisourea (S-methylisothiourea hemisulfate salt) was from Sigma-Alderich (St. Louis, Mo.). Poroszyme immobilized trypsin was from Applied Biosystems (Framingham, Mass.).

Preparation of protein extracts. The protease-deficient S. cerevisiae yeast strain BJ5460 was grown to late-log phase (OD˜3) at 30° C. and protein whole cell extracts prepared as follows: Cells were harvested, frozen, and mechanically lyzed by grinding in the presence of dry ice. The cells were thawed in lysis buffer (8M urea, 1 mM CaCl₂, 100 mPA Tris-HCL, pH8.5). Insoluble debris was pelleted by a high-speed (20 K×g) spin and the supernatant diluted to 2M urea using digestion buffer (100 mM Ammmonium bicarbonate, pH8.5. 1 mM CaCl2. A bacterial whole cell extract was similarly prepared using the E. coli DH5α strain Human nuclear extracts were prepared using a commercial kit (Pierce), and diluted into digestion buffer.

Tryptic Digestion and Peptide Derivatization. Porozyme immobilized trypsin beads were added to an aliquot of each protein extract at a 1:500 protein ratio and the digests incubated at 30° C. for two days with tumbling. The extracts were aliquoted into two microtubes. Solid O-methylisourea was added to one of the tubes to achieve a final concentration of 1M. Base (NaOH) was added to 0.5N to adjust the pH to >10. The reaction was incubated at 37° C. overnight. The peptide mixtures were extracted by solid-phase extraction using SPEC-PLUS PTC18 cartridges (Ansys Diagnostics, Lake Forest, Calif.) according to the manufacturer's instructions and buffer exchanged into a 5% ACN, 0.1% formic acid solution. Samples not immediately analyzed were stored at −80° C.

MCAT peptide sequencing. Each sample was subjected to microcapillary LC-MS/MS analysis with modifications to the general method described by Link and coworkers (1999). A quaternary Surveyor HPLC pump (ThermoFinnigan Canada) was directly coupled to a Finnigan LCQ-DECA ion trap mass spectrometer equipped with a custom microLC eloctrospray ionization source. A fused-silica microcapillary column (100 μm i.d.×365 μm i.d.) was pulled with a Model P-2000 laser puller (Sutter Instrument Co., Novato, Calif.) as described. The microcolumn was packed with 10 cm of 5 μm C_{1B} reversphase material (Zorbax XDB-C18, Hewlett-Packard). Approximately 100 μg of the unmodified fraction and 100 μg of the derivatized peptide fraction were combined and loaded onto a single microcolumn for sequence analysis. After loading, the column was placed in-line with the ion source system setup as described (Link et al, 1999). A fully automated 30 min 100% buffer A (5% ACN, 0.1% formic acid) to 80% solvent B (95% ACN, 0.1% formic acid) binary gradient was run at a flow rate of ˜0.3 ul/min. Eluted peptides were analyzed by automated MS/MS as described by Link and coworkers (1999) except that a full scan range of 400-1600 m/z was used.

SEQUEST analysis. The SEQUEST algorithm (Eng et al., 1994) was run on each dat set against sequence databases obtained from the National Center for Biotechnology Information (Bethesda, Md.). Positive sequence identification was based on several criteria (XCorr and DCn score, and the presence of tryptic termini) described at http, and all identifications were confirmed manually.

MCAT protein quantitation. Pairs of samples to be compared were subjected to automated uLC-MS/MS analysis with modifications to the general method described above. Approximately 200 μg of the unmodified fraction and 200 μg of the derivatized peptide fraction were combined and loaded onto a microcolumn. After loading, a fully automated 30 or 60 min 0-80% A:B gradient chromatography run was carried out on each sample. The buffer solutions used for the chromatography were 5% ACN/0.1% Formic acid (buffer A), 80% ACN/0.1% Formic acid (buffer B). Eluting peptides were analyzed by coupled automated uLC-MS-MS/MS techniques as described above. There was a consistent slight temporal difference in the elution of unmodified/modified peptide, pairs, with the unmodified light analog eluting slightly before the heavy form. Selected ion traces for each peptide pair were quantified using the ADDXPRESS program by which the peak area of each eluting peptide was reconstructed and used in the ratio calculation.

TABLE 1
De novo peptide sequencing from complex mixtures using MCAT

| Identified | b-ion series^{a} | | | b*-ion series^{a} | | | | y-ion series^{b} | | |
|---|---|---|---|---|---|---|---|---|---|---|
| peptide | Expected m/z | Observed m/ | Match^{c} | Expected m/z | Observed m/z | Match^{c} | Δb^{d} | Expected m/z | Observed m/z | Match^{c} |
| Yeast | 717.9 | | | 717.0 | | | | 748.8 | 748.8 | |
| YGR912C | 831.0 | 831.6 | | 831.0 | 831.6 | [symbol] | 0.0 | 006.0 | 886.3 | [symbol] |
| VINDAFGIE | 560.1 | | | 960.1 | | | | 985.1 | 585.4 | [symbol] |
| EGLMTTVHS | 1089.2 | | | 1089.2 | 1089.2 | [symbol] | | 1086.2 | 1086.4 | [symbol] |
| LTATQK | 1146.2 | | | 1146.2 | 1146.2 | [symbol] | | 1187.3 | 1187.6 | [symbol] |
| (SEQ. ID. | 1259.4 | | | 1259.4 | | | | 1318.5 | 1318.3 | [symbol] |
| NO: 16) | 1390.6 | | | 1390.6 | | | | 1431.7 | 1431.7 | [symbol] |
| m = 2575.9 | 1491.7 | 1491.9 | [symbol] | 1491.7 | 1491.0 | [symbol] | 0.1 | 1489.7 | 1489.0 | [symbol] |
| z = 2 | 1597.8 | | | 1592.8 | | | | 1617.8 | 1617.9 | [symbol] |
| | 1691.9 | | | 1691.9 | | | | 1747.0 | 1717.4 | [symbol] |
| | 1829.1 | | | 1829.2 | 1029.1 | [symbol] | | 1860.1 | | |
| | 1916.1 | 1916.3 | [symbol] | 1916.1 | 1916.3 | [symbol] | 0.0 | 1917.2 | 1917.3 | [symbol] |
| E. coli | 340.5 | 340.5 | [symbol] | 340.5 | 340.5 | [symbol] | 0.0 | 317.4 | | |
| R858 | 453.6 | 453.6 | [symbol] | 453.6 | 453.5 | [symbol] | 0.1 | 431.5 | | |
| ILLINPTDSD | 567.7 | 567.3 | [symbol] | 567.7 | 567.3 | [symbol] | 0.0 | 488.5 | 489.4 | [symbol] |
| AVGNAVK | 664.9 | | | 664.9 | 665.4 | [symbol] | | 587.7 | 587.5 | [symbol] |
| (SEQ. ID. | 766.0 | 766.2 | [symbol] | 766.0 | | | | 658.8 | 658.2 | [symbol] |
| NO: 17) | 881.1 | | | 081.1 | 890.7 | | | 773.8 | 773.6 | [symbol] |
| m = 1740.0 | 968.1 | | | 968.1 | | | | 860.9 | | |
| z = 2 | 1083.2 | | | 1083.2 | | | | 976.0 | 975.4 | [symbol] |
| | 1151.3 | 1154.3 | [symbol] | 1154.3 | | | | 1077.1 | 1077.5 | [symbol] |
| | 1253.4 | 1253.5 | [symbol] | 1253.4 | 1253.3 | [symbol] | 0.2 | 1174.2 | 1174.5 | [symbol] |
| | 1310.5 | | | 1310.5 | | | | 1200.3 | 1288.5 | [symbol] |
| | 1424.5 | 1424.6 | [symbol] | 1424.6 | 1424.0 | | 0.6 | 1401.5 | 1401.6 | [symbol] |
| | 1495.7 | | | 1495.7 | | | | 1514.7 | 1514.1 | [symbol] |
| | 1594.8 | 1594.6 | [symbol] | 1594.8 | 1594.6 | | 0.0 | 1677.8 | | |
| HumanACT | 526.6 | | | 526.6 | | | | 568.7 | 568.3 | [symbol] |
| B | 663.7 | 663.4 | [symbol] | 663.7 | 663.4 | [symbol] | | 639.7 | 639.4 | [symbol] |
| VAPEEHPVL | 760.8 | 760.8 | | 760.8 | | | | 768.9 | 768.6 | [symbol] |
| LTEAPLNPK | 859.9 | | | 859.9 | 859.6 | [symbol] | | 870.0 | 869.4 | [symbol] |
| (SEQ. ID. | 973.1 | | | 973.1 | 972.5 | [symbol] | | 983.1 | 983.4 | [symbol] |
| NO: 18) | 1086.3 | 1086.3 | [symbol] | 1086.3 | 1006.5 | [symbol] | 0.7 | 1096.3 | 1095.5 | [symbol] |
| m = 1954.3 | 1187.4 | | | 1187.4 | | | 0.0 | 1195.4 | 1195 | [symbol] |
| z = 2 | 1316.5 | 1315.4 | [symbol] | 1316.5 | 1316.5 | [symbol] | 1.1 | 1292.5 | 1292.6 | [symbol] |
| | 1387.6 | 1387.4 | [symbol] | 1387.6 | 1387.5 | [symbol] | 0.1 | 1429.7 | 1429.7 | [symbol] |
| | 1484.7 | 1484.3 | [symbol] | 1484.7 | | | | 1558.8 | | |
| | 1597.8 | 1597.5 | [symbol] | 1597.8 | 1597.8 | [symbol] | 0.3 | 1687.9 | 1687.7 | [symbol] |
| | 1711.9 | 1711.5 | [symbol] | 1711.9 | 1711.6 | [symbol] | 0.1 | 1785.0 | | |

| Identified | y*-ion series^{b} | | | | | Predicted | |
|---|---|---|---|---|---|---|---|
| peptide | Expected m/z | Observed m/z | Match^{c} | Δy^{e} | Δ(y, y + 1)^{f} | AA^{g} | SEQUEST^{h} |
| Yeast | 790.8 | 791.0 | | 42.2 | 137.0 | II | [symbol] |
| YGR912C | 928.0 | 928.0 | [symbol] | 41.7 | 99.7 | V | [symbol] |
| VINDAFGIE | 1027.1 | 1027.7 | [symbol] | 42.3 | 101.1 | I | [symbol] |
| EGLMTTVHS | 1128.2 | 1120.8 | [symbol] | 42.4 | 100.5 | T | [symbol] |
| LTATQK | 1229.3 | 1229.3 | [symbol] | 42.7 | 131.3 | M | [symbol] |
| (SEQ. ID. | 1360.5 | 1360.6 | [symbol] | 42.3 | 113.3 | I/I | [symbol] |
| NO: 16) | 1473.7 | 1473.9 | [symbol] | 42.2 | 57.2 | G | [symbol] |
| m = 2575.9 | 1530.7 | 1531.1 | [symbol] | 42.1 | 129.0 | E | [symbol] |
| z = 2 | 1659.8 | 1650.1 | [symbol] | 41.2 | 129.2 | F | [symbol] |
| | 1789.0 | 1789.3 | [symbol] | 41.9 | | | |
| | 1902.1 | | | | | | |
| | 1959.2 | 1959.4 | [symbol] | 42.1 | | | |
| E. coli | 359.4 | | | | | | |
| R858 | 473.5 | | | | | | |
| ILLINPTDSD | 530.5 | 530.3 | [symbol] | 40.9 | | | |
| AVGNAVK | 629.7 | 629.4 | [symbol] | 41.9 | 99.1 | V | [symbol] |
| (SEQ. ID. | 700.8 | | | | | | |
| NO: 17) | 815.0 | | | | | | |
| m = 1740.0 | 902.9 | 903.5 | [symbol] | | | | |
| z = 2 | 1010 | 1018.4 | [symbol] | 41 | 114.9 | D | [symbol] |
| | 1119.1 | 1119.6 | [symbol] | 47.1 | 101.2 | T | [symbol] |
| | 1216.2 | 1216.5 | [symbol] | 42.0 | 96.9 | P | [symbol] |
| | 1330.9 | 1330.5 | [symbol] | 42.0 | 114.0 | N | [symbol] |
| | 1443.5 | 1443.5 | [symbol] | 41.9 | 113.0 | I | [symbol] |
| | 1556.7 | | | | | | |
| | 1669.8 | | | | | | |
| HumanACT | 610.7 | 610.7 | [symbol] | 42.4 | | P | |
| B | 681.7 | 681.7 | [symbol] | 42.3 | 71.0 | A | [symbol] |
| VAPEEHPVL | 810.9 | 810.5 | [symbol] | 41.9 | 128.6 | E | [symbol] |
| LTEAPLNPK | 912.0 | 911.5 | [symbol] | 42.1 | 101.0 | T | [symbol] |
| (SEQ. ID. | 1025.1 | 1025.1 | [symbol] | 41.7 | 113.6 | L/I | [symbol] |
| NO: 18) | 1130.3 | 1130.6 | [symbol] | 43.1 | 110.5 | L | [symbol] |
| m = 1954.3 | 1237.4 | | [symbol] | | | | |
| z = 2 | 1334.5 | 1334.5 | [symbol] | 41.9 | | | |
| | 1471.7 | 1471.7 | [symbol] | [symbol] | 137.2 | H | [symbol] |
| | 1600.8 | 1600.4 | [symbol] | | 128.7 | F | [symbol] |
| | 1729.9 | | | | | | |
| | 1827.0 | | | | | | |

^{a}b and b* refer to unmodified and modified b-ion series respectively
^{b}y and y* refer to unmodified and modified y-ion series respectively
^{c} [symbol] indicates a match between expected and observed m/z values (tolerance of 2.0 m/z units)
^{d}Δb, Difference between observed b and b* m/z values
^{e}Δy, Difference between observed y and y* m/z values
^{f}Δ(y, y + 1), Difference in observed m/z between successive y series ions, adjusted for charge state of ion
^{g}Predicted AA, Amino acid residue predicted using Δ(y, y + 1)
^{h} [symbol] indicates a match between MCAT-predicted and SEQUEST-predicted amino acid.

TABLE 2
Identifcation and quantitation of peptides from a yeast whole cell digest.

| | | | | | | | | | | Quantitation^{d} | | |
|---|---|---|---|---|---|---|---|---|---|---|---|---|
| | | | | | | Identification^{a} | | | | Measured | | |
| | | | | | | −MCAT | | +MCAT | | Abundance | | % |
| Protein | Peptide | m^{a} | z | m/z^{b} | Score^{c} | P | P* | P | P* | P | P* | error |
| YBR118W | SVEMHHCQLEQGVPGDNV | 2550.8/ | 2 | 1276.4/ | 2.2433/ | | X | X | [symbol] | 1.00 | 0.76 | 24 ± 4 |
| | CFNVK | 2592.8 | | 1297.4 | 2.5321 | | | | | | | |
| | (SEQ. ID. NO: 19) | | | | | | | | | | | |
| | TLLEAIDAIEQPSRPTDKPL | 3320.8/ | 3 | 1107.9/ | 3.3888/ | [symbol] | X | X | [symbol] | 1.00 | 0.63 | 37 ± 5 |
| | RLPLQDVYK# | 3404.8 | | 1135.9 | 3.3370 | | | | | | | |
| | (SEQ. ID.N0: 20) | | | | | | | | | | | |
| | VETGVIKPGMVVTFAPAGV | 2430.9/ | 2 | 1216.4/ | 2.5458/ | [symbol] | X | X | [symbol] | 1.00 | 0.38 | 62 ± 12 |
| | TTEVK# | 2472.9 | | 1237.4 | 2.1831 | | | | | | | |
| | (SEQ. ID. NO: 21) | | | | | | | | | | | |
| YCR012W | ALENPTAPPLAILGGAK | 1768.1/ | 2 | 885.0/ | 1.7773/ | [symbol] | X | X | [symbol] | 1.00 | 0.57 | 43 ± 5 |
| | (SEQ. ID. N0: 22) | 1810.1 | | 906.0 | 1.4083 | | | | | | | |
| YDR155C | HVVFGEVVDGYDIVK | 1675.9/ | 2 | 838.9/ | 3.7988/ | [symbol] | X | X | [symbol] | 1.00 | 0.71 | 29 ± 5 |
| | SEQ. ID. N0: 23) | 1717.9 | | 859.9 | 3.6211 | | | | | | | |
| YDR487C | HGIPLISIFELAQYLK | 1824.2/ | 2 | 913.1/ | 2.1238/ | [symbol] | X | X | [symbol] | 1.00 | 0.86 | 14 ± 1 |
| | (SEQ. ID. NO: 24) | 1866.2 | | 934.1 | 1.6387 | | | | | | | |
| YGR063C | LPAEVVELLPHYKPR | 1761.1/ | 2 | 881.5/ | 2.0444/ | [symbol] | X | X | [symbol] | 1.00 | 0.66 | 34 ± 6 |
| | (SEQ. ID. NO: 25) | 1803.1 | | 902.5 | 1.9730 | | | | | | | |
| YCR192C | INDAFGIEEGLMTTVHSLT | 2476.8/ | 2 | 1239.4/ | 2.9164/ | [symbol] | X | X | [symbol] | 1.00 | 0.52 | 48 ± 28 |
| | ATQK | 2518.8 | | 1260.4 | 4.1100 | | | | | | | |
| | (SEQ. ID. NO: 26) | | | | | | | | | | | |
| | VINDAFGIEEGLMITVHSL | 2575.9/ | 2 | 1288.9/ | 3.1456/ | [symbol] | X | X | [symbol] | 1.00 | 0.44 | 56 ± 17 |
| | TATQK | 2617.9 | | 1309.9 | 3.3717 | | | | | | | |
| | (SEQ. ID. NO: 27) | | | | | | | | | | | |
| | VPTVDVSVVDLTVK | 1512.7/ | 2 | 757.3/ | 3.2279/ | [symbol] | X | X | [symbol] | 1.00 | 1.29 | 29 ± 11 |
| | (SEQ. ID. NO: 28) | 1554.7 | | 778.3 | 3.1548 | | | | | | | |
| YGR214W | NVQVHQEPYVFNARPDGV | 2817.2/ | 3 | 940.0/ | 1.8494/ | [symbol] | X | X | [symbol] | 1.00 | 0.61 | 39 ± 10 |
| | HVINVGK | 2859.2 | | 954.0 | 2.2204 | | | | | | | |
| | (SEQ. ID. NO: 29) | | | | | | | | | | | |
| YGR254W | AQYNEIQGWDHLSLLPTF | 2398.7/ | 2 | 1195.3/ | 2.4748/ | [symbol] | X | X | [symbol] | 1.00 | 0.81 | 19 ± 7 |
| | GAK (SEQ. ID. NO: 30) | 2430.7 | | 1216.3 | 3.0844 | | | | | | | |
| | YPIVSIEDPFAEDDWEAW | 2829.1/ | 3 | 944.0/ | 3.1108/ | [symbol] | X | X | [symbol] | 1.00 | 0.61 | 39 ± 9 |
| | SHFFK | 2871.1 | | 958.0 | 3.2183 | | | | | | | |
| | (SEQ. ID. NO: 31) | | | | | | | | | | | |
| YHR174W | WLTGVELADMYHSLMK | 1894.2/ | 2 | 948.1/ | 4.0552/ | [symbol] | X | X | [symbol] | 1.00 | 0.77 | 23 ± 3 |
| | (SEQ. ID. NO: 32 | 1936.2 | | 969.1 | 3.8246 | | | | | | | |
| YJR105C | TVIFTHGVFPTVVVSSK | 1800.1/ | 2 | 901.0/ | 1.5600/ | [symbol] | X | X | [symbol] | 1.00 | 0.75 | 25 ± 4 |
| | (SEQ. ID. NO: 33) | 1842.1 | | 922.0 | 1.8810 | | | | | | | |
| YKL060C | SPIILQTSNGGAAYFAGK | 1795.0/ | 2 | 898.5/ | 3.6709/ | [symbol] | X | X | [symbol] | 1.00 | 0.73 | 27 ± 5 |
| | (SEQ. ID. NO: 34) | 1837.0 | | 919.5 | 4.2032 | | | | | | | |
| | TGVIVGCDVIINLTYAK | 1863.1/ | 2 | 932.5/ | 3.2735/ | [symbol] | X | X | [symbol] | 1.00 | 0.75 | 25 ± 4 |
| | (SEQ. ID. NO: 35) | 1905.1 | | 953.5 | 2.6813 | | | | | | | |
| YLR044C | KLIDLTQRPAFVTPMGK# | 1906.3/ | 2 | 954.1/ | 3.5845/ | [symbol] | X | X | [symbol] | 1.00 | 0.83 | 17 ± 2 |
| | (SEQ. ID. NO: 36) | 1948.3 | | 975.1 | 3.9361 | | | | | | | |
| YLR058C | EVLYDLENPINFSVFPGHQ | 3772.2/ | 3 | 1258.4/ | 1.8356/ | [symbol] | X | X | [symbol] | 1.00 | 0.73 | 27 ± 6 |
| | GGPHNHTIAALATALK | 3814.2 | | 1272.4 | 2.5693 | | | | | | | |
| | (SEQ. ID. NO: 37) | | | | | | | | | | | |

^{a}Molecular mass of unmodlfied/modified peptides ions.
^{b}Mass-to-charge ratio of unmodified/modified peptides
^{c}SEQUEST cross-correlation score for unmodified/modified peptide.
^{d}Identifications were determined in untreated samples (−MCAT) or samples modified using MCAT (+MCAT). [symbol] or X Indicates that the unmodified (P) or modified (P*) peptides were observed ( [symbol] ) or not observed (X) In the respective sample.
^{e}Relative abundance measurements aRe for 1:1 mixture of unmodified and modified samples. Percentage error refers to deviation from ideal (1:1) ratio ± standard deviation for multiple measurements.
#These peptides were modified at more than one lysine residue.

Further Discussion of the Figures Related to MCAT
(1) The MCAT Approach for Peptide Sequencing and Relative Protein Abundance Determination.

See FIG. 1. (A) The guanidination reaction is specific for the side chains of lysine, which is selectively converted to homoarginine. (B) For sequencing using MCAT, protein mixtures are first digested with trypsin, which generates peptides suitable for MS analysis that terminate with lysine or arginine residues. Half of the sample is treated with the MCAT reagent O-methylisourea, Peptides ending in lysine are modified, which adds 42 amu to the mass of the peptide but does not alter the properties of the peptide during LC-MS analysis. The peptides mixtures are combined at a 1:1 ratio, separated by reverse phase LC and introduced online into a MS instrument using electrospray ionization. Following tandem MS analysis, peptide sequence is determined by comparing MS/MS spectra of unmodified and modified peptides. The fragmentation pattern of both sister peptide pairs are similar except for the shifted y-ion series, which can be deconvoluted to reveal the amino acid sequence of the peptide. (C) For relative abundance measurements, samples representing different cell states are alternatively modified or unmodified with MCAT. Full MS spectra are recorded for sister peptide species and their relative abundance determined by measuring the respective trace intensities on reconstructed single ion chromatograms.

(2) MCAT Enables Identification and Quantitation of Complex Protein Mixtures.

See FIG. 2. (A) Ion chromatograms recorded for the base peak (top), an unmodified peptide ion [LPWFDGMLEADEAYFK+2H]⁺² (middle) and its corresponding O-methylisourea(MCAT)-modified form (bottom). When mixtures of untreated and MCAT-treated protein digests are resolved by reverse phase LC, the modified peptides elute with a minor delay compared to the respective unmodified forms (35.9 vs. 35.7 min respectively in this example). (B) Depending on charge and the number lysine residues, the m/z signals observed for pairs of unmodified or modified peptide ions during MS are offset by 42, 21 or 14 m/z units (for plus 1, 2 or 3 ions respectively). In this example, the peak signals recorded for the unmodified (967.07 m/z) and modified (988.08 m/z) forms of the peptide are offset by 21 m/z units, indicating a +2 charge. The peptide ions are then independently selected and automatically fragmented by MS/MS. Comparison of the y-ion series allows the amino acid sequence to be determined. (C) The relative abundance of individual peptides can be determined by reconstructing the chromatograms for the unmodified and modified forms of the peptide ions and calculating the ratio of signal intensities using area under curve integration.

(3) De Novo Sequencing of a Yeast Peptide and a Human Peptide Using MCAT Approach.

See FIGS. 3A and 3B. (A) The peptide WVDLVEHVAK (SEQ ID NO:38)analyzed by MCAT LC-MS/MS in a digest of yeast whole cell extract. A representative MS/MS spectrum of the unmodified peptide (top) and the corresponding spectrum for the modified form (below) are shown. Because the MCAT reagent reacts specifically with lysine residues, the carboxy-terminal lysine of a tryptic peptide is uniquely modified. Therefore, the signals for the y-series of ions (where charge localizes to the carboxy-terminal lysine) are shifted+42 m/z units and can be immediately identified, whereas the b-series of ions (where charge is retained at the amino terminus) are unaltered. The expected m/z values for b- and y-series ions of the unmodified and modified peptides are given (right), with those observed in the experiment underlined. The amino acid order is resolved by measuring the mass difference between successive y-ion peaks. (B) The peptide VAPEEHPVLLTEAPLNPK (SEQ ID NO:39)was identified in a digest of nuclear extract from HeLa cells. In this peptide a stretch of ten amino acids (A-E-T-L/I—U/I—V—P—H-E-E) can be identified by mapping y-ions to the bands shifted by 42 m/z units in the modified spectrum (bottom) relative to the unmodified spectrum (top). The dominant peak at 892.9 in the unmodified spectrum is approximately 21 m/z units from an dominant unassigned peak at 914.4 in the modified spectrum. These peaks probably represent doubly-charged y16 ions that terminate in with proline, an amino acid commonly observed to form dominant peaks during CID. The other major peak in both spectra (1292.6 and 1334.5 in the upper and lower panels respectively) is a singly-charged y12 ion that also terminates with proline. Therefore, an additional advantage of the MCAT is technique is the resolution of such ambiguous peaks through charge determination. In the case of both yeast and human peptides, the identical molecular masses of leucine and isoleucine prevent their resolution by MS.

(4) The MCAT Method is Reproducible and Quantitative.

See FIGS. 4A and 4B. (A) A yeast whole cell was digested with trypsin in three replicate experiments (A, B, C). Each digest was divided into two equal portions, one of which was treated with O-methylisourea. Each pair of mixtures was then recombined at a 1:1 ratio and protein quantitation determined by the MCAT LC-MS/MS. The relative abundance ratios (expressed at the ratio of modified to unmodified peptide signal) of a subset of positively-identified peptides is given for each analysis. (B) Untreated and MCAT-labeled yeast protein tryptic digests were combined in varying proportions ranging from from 16:1 (modified to unmodified) to 1:16 effective concentrations. The measured relative abundance ratios for five representative peptides are plotted versus the log(10) of the dilution ratio.

Peptide Profiling

Below examples are shown of the utility of peptide profiling as a means to characterize and classify diverse human tissues, to characterize subcellular fractions of individual tissues, and to illustrate how a database of such peptide profiles can serve as a depository of protein expression information that can be mined rapidly and accurately for knowledge about the status of an unknown sample. This process is robust, sensitive and reproducible. Although the method is generally applicable, the following serve to illustrate select uses of the approach.

### Example 3

### Use of Peptide Profiles to Characterize Human Tissue

The invention includes methods of characterizing human tissue. The method comprises generating samples suitable for MS analysis and producing a peptide profile. The relative abundance of peptides in samples is also preferably determined. The peptide profile that is generated is compared to peptide profiles in a database or library using common algorithms in order to identify cognate proteins, preferably those that are considered important therapeutic targets, as well as metabolic enzymes and structural proteins.

Table 1 shows 40 peptides sequenced and quantified from a human lung tissue lysate sample in a single LC-MS analysis that are then used to construct a unique peptide profile. The peptides in turn allowed for the identification of cognate corresponding proteins present in the sample (a total of 867 proteins were unambiguously identified in this analysis). Note that the peptides sequences obtained by a generic database search algorithm were both preceded by, and terminated with, a K or R residue as a result of cleavage of the input proteins by trypsin. The sequence of a total of 1896 peptides were determined in this one analysis with high accuracy and sensitivity, demonstrating the ability of the approach to generate a detailed profile or fingerprint of protein expression of a complex tissue.

TABLE 1

Partial List of Peptides observed in human
lung tissue used for peptide profiling.

| | |
|---|---|
| K.AAIANLCIGDLITAIDGEDTSSMTHLEAQ | (SEQ. ID. NO:40) |
| NK.I | |
| K.AALAGGTTMIIDHVVPEPGTSLLAAFDQWR.E | (SEQ. ID. NO:41) |
| K.AAPLSLCALTAVDQSVLLLKPEAK.L | (SEQ. ID. NO:42) |
| K.AAQAHEDIIHGSGK.T | (SEQ. ID. NO:43) |
| K.AASLGSSQPSRPHVGEAATATK.V | (SEQ. ID. NO:44) |
| K.AASWLTHQGSFHGAFR.S | (SEQ. ID. NO:45) |
| K.AAVFNHFISDGVKK.T | (SEQ. ID. NO:46) |
| K.AAVLWELHKPFTIEDIEVAPPK.A | (SEQ. ID. NO:47) |
| K.AAVSGLWGK.V | (SEQ. ID. NO:48) |
| K.ACISPKPQKPWDK.D | (SEQ. ID. NO:49) |
| K.ADIIYPGHGPVIHNAEAK.I | (SEQ. ID. NO:50) |
| K.AEEVAFWTELLAK.N | (SEQ. ID. NO:51) |
| K.AEGPEVDVNLPK.A | (SEQ. ID. NO:52) |
| K.AFAMIIDKLEEDISSSMTNSTAASRPPVT | (SEQ. ID. NO:53) |
| LR.L | |
| K.AFAQAQSHIFIEK.T | (SEQ. ID. NO:54) |
| K.AFISNVKTALAATNPAVR.T | (SEQ. ID. NO:55) |
| K.AGAFCLSEDAGLGISSTASLR.A | (SEQ. ID. NO:56) |
| K.AGAPPGLFNVVQGGAATGQFLCHHR.E | (SEQ. ID. NO:57) |
| K.AGHPFMWNEHLGYVLTCPSNLGTGLR.G | (SEQ. ID. NO:58) |
| K.AGNNMLLVGVHGPR.T | (SEQ. ID. NO:59) |
| K.AHGPGLEGGLVGKPAEFTIDTK.G | (SEQ. ID. NO:60) |
| K.AHSPQGEGEIPLHR.G | (SEQ. ID. NO:61) |
| K.AHVSFKPTVAQQR.I | (SEQ. ID. NO:62) |
| K.AIEVIRPAHILQEK.E | (SEQ. ID. NO:63) |
| K.AIQDAGCQVLK.C | (SEQ. ID. NO:64) |
| K.AKFENLCK.L | (SEQ. ID. NO:65) |
| K.AKPVVSFIAGITAPPGR.R | (SEQ. ID. NO:66) |
| K.ALEHSALAINHK.L | (SEQ. ID. NO:67) |
| K.ALESPERPFLAILGGAK.V | (SEQ. ID. NO:68) |
| K.ALGGIGPVDLLVNNAALVIMQPFLEVTK.E | SEQ. ID. NO:69) |
| K.ALHASGAK.V | (SEQ. ID. NO:70) |
| K.ALHASGAKVVAVTR.T | (SEQ. ID. NO:71) |
| K.ALLNNSHYYHMAHGK.D | (SEQ. ID. NO:72) |
| K.ALNRPPTYPTK.Y | (SEQ. ID NO:73) |
| K.ALPGHLKPFETLLSQNQGGK.A | (SEQ. ID. NO:74) |
| K.ALSDHHVYLEGTLLKPNMVTPGHACTQK.F | (SEQ. ID. NO:75) |
| K.ALTGGIAHLFK.Q | (SEQ. ID. NO:76) |
| K.ALVKPQAIKPK.M | (SEQ. ID. NO:77) |

A further embodiment of the invention includes using profiles such as this to compare different tissues or experimental samples. For instance, a comparison of the peptide profiles for human pancreatic and heart tissues can be made with a simple 2-dimensional plot that can be extended to ‘n’ different planes as required (for ‘n’ types of tissue, samples, or patients). Comparison of the peptide profiles of these samples can be done using standard computational methods (e.g. agglomerative clustering). In the case of human pancreatic tissue, the analysis showed that although several proteins are shared between the tissues, many are not. Therefore, a further embodiment of the invention is the use of peptide profiles to characterize tissues and thereby categorize samples.

Although this patent describes primarily approaches involving peptide profiling, the approach can be extended to whole protein profiling (and to other applications where separation techniques compatible with mass spectrometry may be used to elicit a profile, for instance lipid profiling, phasphoproteins profiling, small molecule metabolite profiling; these methods preferably involve tagging the compounds of interest and performing LC-MCAT to generate a lipid profile, phosphoprotein profile, small molecule metabolite profile. The methods can provide identity and relative abundance information by readily adapting the methods described herein with peptides).

Table 2 shows some of the corresponding proteins (of the 867 unique proteins identified in this analysis) identified by searching the SwissProt Protein database using the identified peptide sequences (http://www.expasy.ch/sprot/).

TABLE 2
Proteins identified using peptides isolated from human lung tissue.

| | |
|---|---|
| P47915 | 60 s ribosomal protein I29, 5/2000 |
| | [MASS = 17456] |
| P48025 | tyrosine-protein kinase syk (ec 2.7.1.112) |
| | (spleen tyrosine kinase). 11/1997 |
| P48147 | prolyl endopeptidase (ec 3.4.21.26) |
| | (post-proline cleaving enzyme) (pe). 10/1 |
| P48444 | coatomer delta subunit (delta-coat protein) |
| | (delta-cop) (archain). 11/1997 [M |
| P48634 | large proline-rich protein bat2 (hla-b-associated |
| | transcript 2). 2/1996 [MASS |
| P48735 | isocitrato dehydrogenase [nadp], mitochondrial |
| | precursor (ec 1.1.1.42) (oxalo |
| P49023 | paxillin. 7/1998 [MASS = 60937] |
| P49137 | map kinase-activated protein kinase 2 (ec 2.7.1.-) |
| | (mapK-activated protein ki |
| P49182 | heparin cofactor ii precursor (hc-ii) (protease |
| | inhibitor leusorpin 2) 11/19 |
| P49321 | nuclear autoantigenic sperm protein (nasp). |
| | 7/1998 [MASS = 65191] |
| P49327 | fatty acid synthase (ec 2.3.1.85) [includes: |
| | ec 2.3.1.38; ec 2.3.1.39; ec 2.3 |
| P49407 | beta-arrestin 1. 7/1999 [MASS = 46969] |
| P49411 | elongation factor tu. mitochondrial precursor |
| | (p43). 12/1998 [MASS = 49542] |
| P49773 | hint protein (protein kinase c inhibitor 1) |
| | (pkci-1). 7/1998 [MASS = 13671] |
| P50096 | inosine-5′-monophosphate dehydrogenase 1 |
| | (oc 1.1.1.205) (imp dehydrogenase 1) |
| P50552 | vasodilator-stimulated phosphoprotein |
| | (vasp). 11/1997 [MASS = 39830] |
| P50748 | hypothetical protein klaa0166. 11/1997 |
| | [MASS = 260749] |
| P50651 | cdc4-like protein (fragment). 7/1998 |
| | [MASS = 213599] |
| P51174 | acyl-coa dehydrogenase. long-chain specific |
| | precursor (ec 1.3.99.13) (icad). |
| P51660 | estradiol 17 beta-dehydrogenase 4 (ec 1.1.1.62) |
| | (17-beta-hsd 4) (17-beta-hydr |
| P51790 | chloride channel protein 3 (clc-3). 7/1998 |
| | [MASS = 64793] |
| P51812 | ribosomal protein s6 kinsse ii alpha 3 |
| | (ec 2.7.1.-) (s6kii-alpha 3) (p90-rsk |
| P51885 | lumicen precursor (lum) (keralan sulfate |
| | proteoglycan). 7/1998 [MASS = 38351] |
| P51981 | heterogeneous nuclear ribonucleoprotein a3 |
| | (hnrnp a3) (fbrnp) (d10s102). 7/19 |
| P52272 | heterogeneous nuclear ribonucleoprotein m |
| | (hnrnp m). 10/1996 [MASS = 77469] |
| P52480 | pyruvate kinase, m2 isozyme (ec 2.7.1.40). |
| | 7/1999 [MASS = 57756] |

Cursory examination of this list shows that many interesting and therapeutically important proteins are identified by this process, including low abundance regulatory proteins such as signaling proteins, transport channels, and nuclear proteins.

A common criticism of current proteomics technologies based on two-dimensional polyacrylamide gels is that they are insensitive and only identify high abundance metabolic proteins, ie. proteins that are not normally critical determents of disease (although these can be important effectors of disease) especially since drug development strategies nearly always target low abundance proteins important for counteracting a disease phenotype.

It is clear from the above table that peptide profiling can successfully describe many proteins that are considered important therapeutic targets, and not just metabolic enzymes and structural proteins.

Table 3 shows how proteins from various therapeutically important categories were readily identified and quantified in a single analysis. This list was made using keywords present in the sequence annotation databases and therefore represents the minimum representation of such classes—the vast majority of sequenced mammalian proteins await functional annotation.

By contrast, a recently published study (Proteomics 1,1303-19 A database of protein expression in lung cancer.Oh J M, Brichory F. Puravs E, Kulck R. Wood C, Roulllard J M, Tra J, Kardia S, Beer D, Hanash S. 2001) where over 1300 2D gels were analyzed from a variety of different lung cell lines and tumors, identified less than 200 proteins, the majority of which were metabolic and structural proteins of high abundance, and provided no quantitative information.

TABLE 3
Peptide profiling identifies therapeutically important proteins.

| | | Conventional |
|---|---|---|
| | Peptide | approach |
| | profiling | (Oh et al) |
| Kinases | 46 | 1 |
| Phosphatases | 12 | 1 |
| Integrins | 9 | 0 |
| Channel proteins | 12 | 0 |
| Apoptosis proteins | 1 | 0 |
| Proteins contributing | 10 | 0 |
| to cancer | | |
| Proteins with homology | 27 | 0 |
| to viral proteins | | |
| Antigenic | 22 | 4 |
| p53-related proteins | 7 | 0 |
| MHC proteins | 4 | 1 |
| Cytokines and interleukins | 14 | 0 |

### Example 4

### Peptide profiling to Characterize Diverse Human Tissues

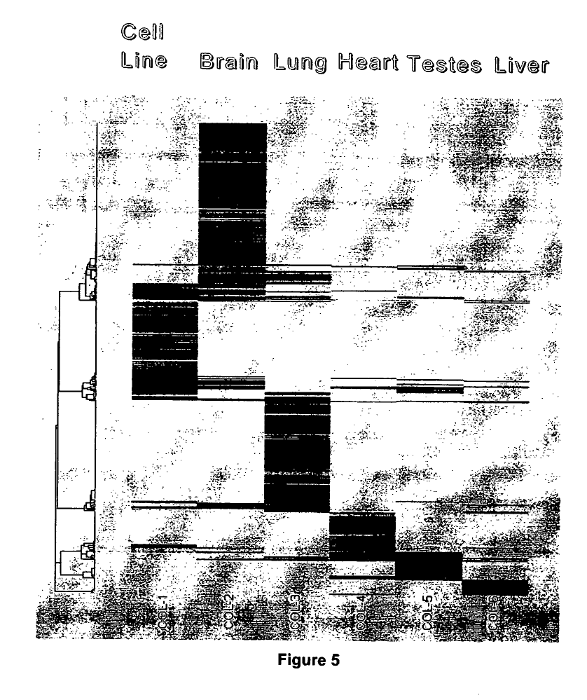

One-dimensional LCMS was used to obtain peptide profiles from diverse human tissues (FIG. 5). The one-dimensional approach has 2- to 10-fold lower resolution compared to two-dimensional approaches but was used in this case to example a large number of samples to illustrate the principle. Table 4 shows the number of peptides and proteins identified for different human tissues.

TABLE 4
The peptide profiling approach can be applied to diverse tissues.

| | Proteins | Peptides |
|---|---|---|
| Brain | 359 | 734 |
| Heart | 114 | 231 |
| Testes | 78 | 136 |
| Liver | 56 | 83 |
| Muscle | 72 | 66 |
| Plasma | 288 | 846 |
| Pancreas | 202 | 283 |

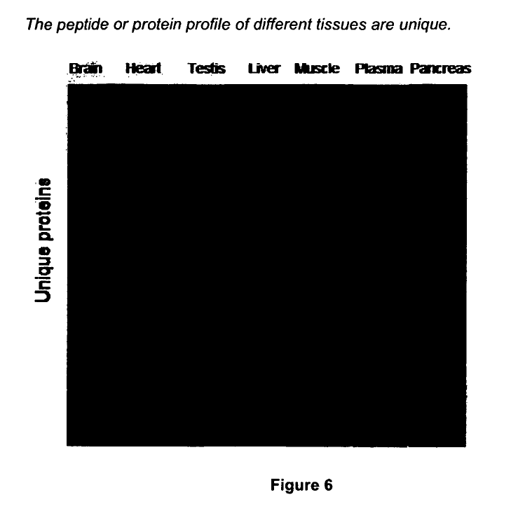

It is assumed that diverse tissues may express many similar proteins (for instance ribosome associated proteins), yet express a subset of unique proteins that functionally distinguishes one tissue from another. Similarly, the proteome of diseased tissue may be different to healthy tissue. Although this malt seem self-evident, very few studies have addressed these issues by directly comparing the proteomes from different samples. This is largely because of the technical impediments mentioned above—conventional techniques generally characterize only the most abundant proteins and peptides, and these peptides are least likely to differ from tissue to tissue. FIG. 6 shows how many proteins were identified using MCAT based peptide profiling for a preliminary study of seven human tissues. Notably, the peptide and protein profiles of each tissue is distinct. Even with this preliminary low resolution analysis, each tissue evokes a different signature when subjected to peptide profiling.

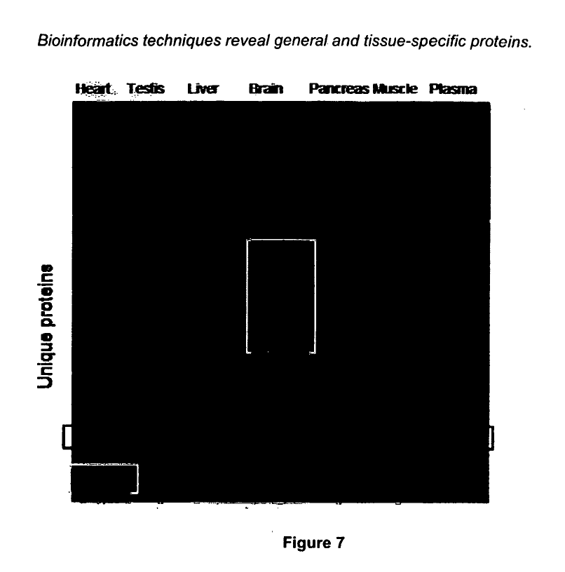

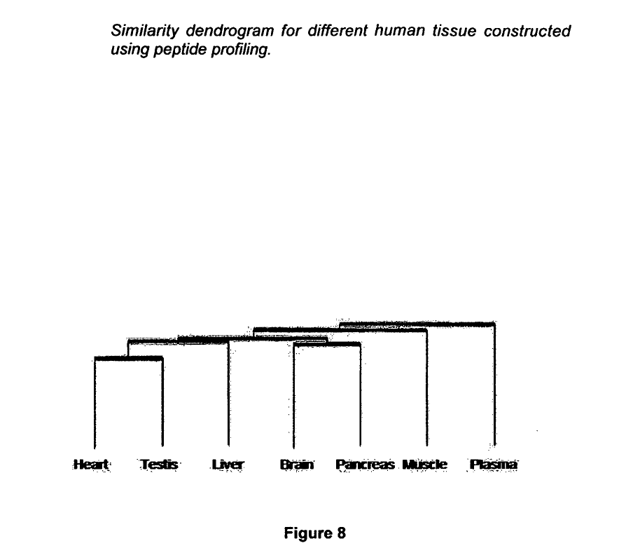

When the proteins identified for different tissues are compared, it is clear that some proteins are common to several tissues, while some are tissue-specific (FIG. 6). These differences can be highlighted by applying agglomerative clustering algorithms to the data (FIG. 7). In this figure as an example, common proteins are highlighted in the large rectangular box, while heart- and brain-specific proteins are highlighted in the smaller rectangular boxes. Furthermore, the degree of relationship between these tissues can be established by comparing such peptide profiles (FIG. 8). Although the principle was illustrated here using different human tissues, such analysis can be used to detect other proteomic changes, for instance human heart tissue following exercise or myocardial infarction, or following administration of drugs.

### Example 4

### Peptide Profiling to Characterize Subcellular Fractions of a Single Tissue

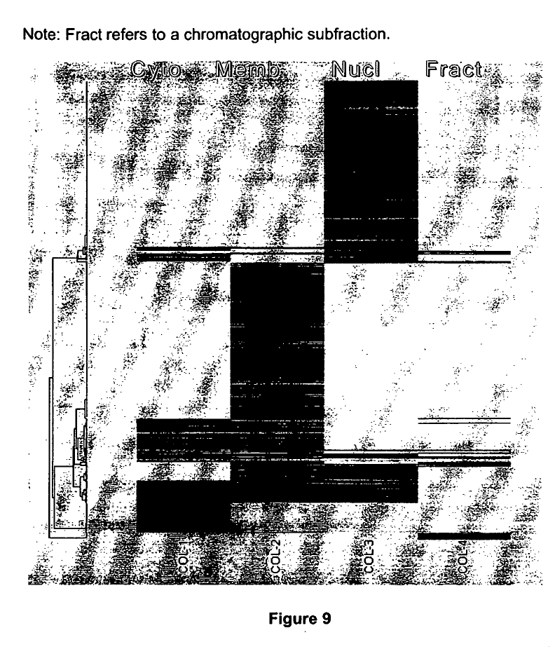

In another embodiment of the invention, peptide profiling can be used to analyze the subtractions of a cell, preferably into nuclear, cytoplasmic and membrane fractions. This discriminatory power of peptide profiling is illustrated here, where the method is used to examine the subfractions of a single clonal cell line. Cultured human myoblast cells were processed into nuclear, cytoplasmic and membrane fractions and analyzed using the peptide profiling technique (FIG. 9). Significantly, over 400 membrane-localized proteins were identified. This class is normally very difficult to analyze using conventional proteomics methods yet is of particular pharmacologic/therapeutic interest, being the site of receptors and channels with critical signaling and transport functions.

Tables 7 and 8 show how peptide profiling can be applied to different cellular subfractions and used to identify compartment-specific proteins.

TABLE 7
Peptide profiling applied to different cell compartments.

| | Peptides | Proteins |
|---|---|---|
| Cytoplasmic | 2220 | 994 |
| Nuclear | 804 | 428 |
| Membrane | 727 | 403 |

TABLE 8
Peptide profiling identifies compartment-specific proteins

| | Cytoplasmic | Membrane | Nuclear |
|---|---|---|---|
| Unique | 805 | 249 | 262 |
| Total | 994 | 428 | 403 |
| Percent | 80 | 58 | 65 |
| unique | | | |

### Example 5

### Use of Peptide Profiles to Characterize Human Cell Lines

In another embodiment of the invention, this invention includes methods of characterizing human cell lines. The method comprises generating samples suitable for MS analysis and producing a peptide profile. The relative abundance of peptides in samples is also preferably determined. The peptide profile that is generated is compared to peptide profiles in a database or library using common algorithms in order to identity cognate proteins, preferably those that are considered important therapeutic targets, as well as metabolic enzymes and structural proteins. In a further embodiment, these profiles can comprise a small prototype database or library, against which novel samples may be screened.

A number of peptides from four human cell lines of distinct cellular origin are identified by mass spectrometry and linked to their parent proteins. This profile is one-dimensional because no addition information about the peptides. (e.g. quantitative information) is included. Table 6 shows the number of peptides and proteins identified for the different human cell lines.

TABLE 6
Peptide profiling of different cultured cells

| | Proteins | Peptides |
|---|---|---|
| Myoblasts | 576 | 1373 |
| HeLa | 974 | 2067 |
| NYP17 | 192 | 290 |
| Raji-Jurkat | 233 | 376 |

Here, an independent extract of one of the four cell lines is screened and demonstrates how this extract can be conclusively shown to be highly similar or identical to a profile in the database.

Method

Cell extracts derived from four human cell lines (MCF7, TPA, Jurkat, K566) were digested with trypsin (Porozyme, Perceptive Biosystems, USA) and analyzed using an ion trap mass spectrometer (Deca, Thermoquest, USA) following separation of digested peptides using online HPLC. The mass spectrometer was programmed to collect primary MS spectra from parent ions, as well as tandem mass spectra of daughter ions generated from the first, second and third most abundant ions observed in the program window. These spectra were then used to search nonredundant genome databases using the SEQUEST algorithm (Yates et al., 1995) to identify the peptides and proteins present in the samples.

The following table shows the top-scoring peptides identified in the analysis of one of these cell lines, Jurkat: experiment, After statistical filtering, 74, 91, 96, 123 peptides were used to identify 55, 62, 49, 59 different proteins in the respective cell. The peptides for all four cell lines were deposited into a database, in this case a Microsoft Access file, The protein profiles are graphically represented below (5922, 4091, 5644 and 4166 tryptic peptides were observed from MCF7, TPA, Jurkat and K566 cells respectively, visual representation):

[image]

If these profiles are considered as a small index or database, novel profiles can be searched against them using any common correlation test. For instance here the correlation is calculated by:
P_{x,y}=[1n_{(j=1 to n)}Σ(Xⱼ−μₓ)(Yⱼ−μ_{y})]/[∂ₓ·∂_{y}]
where peptides common to two profiles score ‘1’ and peptides not shared between profiles score ‘0’.

Correlation scores, P_{x,y}, for one-dimensional peptide profiles obtained from four human cell lines:

| | MCF7 | TPA | Jurkat | K556 | ? |
|---|---|---|---|---|---|
| MCF7 | 1 | 0.0105 | 0.33596 | 0.09 | 0.07 |
| TPA | 0.0105 | 1 | 0.33596 | 0.31714 | 0.26733 |
| Jurkat | 0.33596 | 0.33595 | 1 | 0.09 | .8644 |
| K556 | 0.09 | 0.31714 | 0.09 | 1 | 0.09 |

This preliminary analysis suggests that the peptide profiles obtained from Jurkat and MCF7, and Jurkat and TPA nuclear extracts are more similar than those obtained for other combinations. More importantly, when the peptide profile obtained from an independent preparation of Jurkat nuclear extract (labeled ‘?’ in the above Table), it received a high score and could be identified as being most closely related to the Jurkat cells.

Applications of Protein Expression Datasets

Relevance to Disease

As an example of the approach, its potential use in the diagnosis and study of human disease is described, for example in infectious disease or a genetic disease such as cancer. The invention may be used to systematically identify, compare, classify, and characterize and investigate biological or clinical samples from normal and virus- or bacterially-infected cells and tissues, similar cells obtained over a course of infection, or similar cells obtained over the course of a therapeutic treatment. Similarly, the invention may be used to systematically identify, compare, classify, and characterize and investigate biological or clinical samples from normal and cancerous cells and tissues, cancerous cells and tissues obtained from a variety of related or unrelated liquid or solid tumors, cells obtained over time that follow the development of a progressive cancer, or cells similarly obtained over time that follow the progression of a therapeutic intervention.

The resulting datasets or profiles may therefore (i) identify robust signatures of disease states that can be used to facilitate diagnostic and prognostic medical procedures, (ii) refine current models of disease and highlight productive areas for focusing further basic and applied investigative approaches.

Uses in Toxicology Studies

As another example of the use of the invention, quantitative peptide profiles may be used for investigation of toxic effects in human or other tissues or cells, for instance the side-effects of candidate drug compounds. This is because the toxicity may be represented by changes in the expression patterns of peptides and proteins in the cells. Currently, such toxic effects are investigated using general marker enzymes such as cytochrome oxidase. In many ways, this is a ‘blunt tool’, failing to differentiate between different types of toxicity, and/or the severity of the toxic effect. Quantitative peptide profiles are likely to be discrete for individual compounds while profiles generated in response to related compounds would be expected to be also related to each other.

A database of profiles can be assembled that describes the protein complements of tissues treated with known toxic agents. Large numbers of drug candidates can then be screened and their profiles compared to those in the reference database. Accordingly, the invention includes methods of determining the toxicity of a candidate drug compound. The method comprises administering the candidate compound to a cell. As described above, samples suitable for MS anaylsis are generated and a peptide profile is produced. Relative abundance of peptides in samples is also preferably determined. This candidate compound peptide profile is compared to peptide profiles in a database or library (for example, profiles showing the cell in a normal state and in varied states of toxicity). If the candidate compound sample profile is highly similar to (for example, greater than 90%, 95%, or 99% similarity), or identical to a profile in the database or library, then that similarity shows the amount of toxicity of the candidate compound to the cell. If the candidate compound sample profile is highly similar to a normal cell profile, then the candidate compound is less likely to be toxic than if the candidate compound sample profile is similar to the peptide profile of the cell in state of toxicity. The relative abundance of the test sample peptides is also preferably compared to other profiles to determine the amount of toxicity of a candidate compound.

Profiles obtained from drug candidates that are similar to those obtained from damaged tissue alert the investigators to potential toxicity problems associated with that compound. Because each single profile comprises a large dataset (many individual proteins and their relative abundances), comparison of the profiles is statistically powerful. This reduces dependence on animal toxicity trials, where large numbers of animals may be necessary to obtain statistically relevant data.

Healthy cells, and cells treated with toxic agents, will be analyzed by liquid chromatography-tandem mass spectrometry (LC-MS/MS) using 2 novel semi-quantitative approach, resulting in a protein profile for each treatment that serves as a signature of the cell state. The profile comprises data relating tens to hundreds of individual proteins and therefore represents a highly specific and sensitive description of the protein complement of the cell or tissue in that particular state.

Even without knowledge of protein function, the profiles from cells treated with novel compounds can be compared to those from healthy cells or cells treated with toxic compounds. The method may therefore be predictive of toxic effects at an early stage of drug development. Further, where the test profile matches the profile produced by treatment with a characterized compound or family of compounds, the mechanism of toxicity may be similar to that produced by the reference class. This application of the invention can be applied to any primary or transformed cell line, or to tissues obtained from animal models, preferably mammalian and more preferably human, or to experimental or clinical samples.

### Example 6

### Peptide Profiling to Characterize the Effects of a Drug on a Tissue

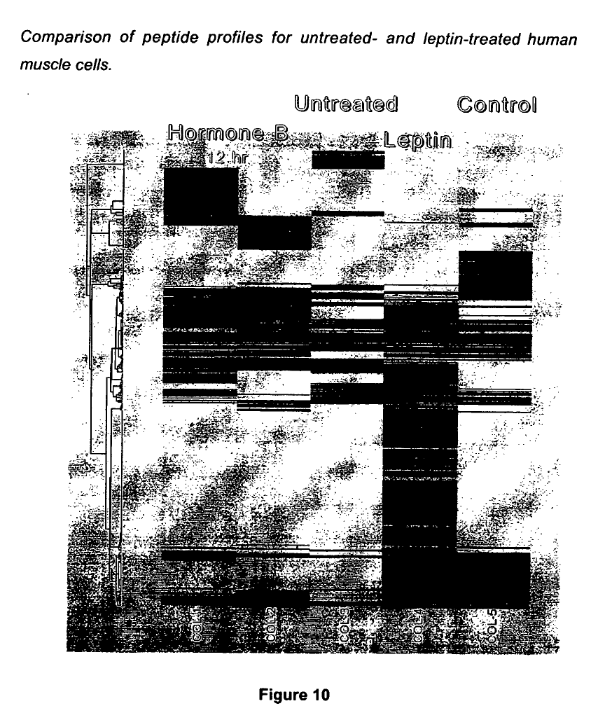

A further embodiment of the peptide profiling invention is to characterize and identify the effect of drugs and other experimental treatments on the proteome. In this example, cultured human muscle cells were treated with the hormone drug leptin. For both treated and untreated samples, over 400 proteins and 900 peptides were identified. Of these, 170 were uniquely observed in one or other sample. In FIG. 10, a screenshot of this analysis shows peptides present in one or other sample (green or red) and peptides unique to either sample (blue). This experiment demonstrates that the invention can be used to examine the effect of drugs and other treatments on proteome mixtures.

### Example 7

### Peptide Profiling to characterize Tissue from Different Organisms

As further proof of principle, the peptide profiling approach was applied to different organisms—two microbes (Escherchia coli and Saccharomyces cerevisiae) and two mammals (Homo sapiens—humans and Mus musculis—common lab mouse). A standard MCAT LC-MS peptide profiling analysis was used to follow expression of hundreds of proteins for each species (Tables 9, 10).

TABLE 9
Peptide profiling of microbial species.

| | Proteins | Peptides |
|---|---|---|
| Yeast | 233 | 519 |
| Bacteria | 542 | 1647 |

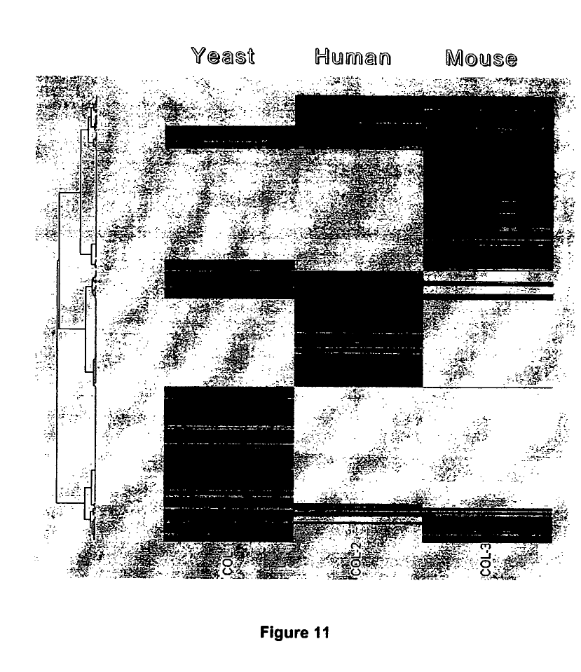

When the peptide profiles of the highly divergent microbial species were compared, 516 of the 519 yeast proteins were unique. In contrast, when a similar analysis was done for peptide profiles of the two mammalian species, 44 of 197 mouse peptides were similarly observed in the human profile (representing homologous protein/peptide species). Thus, these preliminary analyses indicate that peptide profiling can both distinguish species, and that the peptide profile may reflect the degree of relatedness of organisms (FIG. 11).

TABLE 10
Peptide profiling of mammalian species.

| | Proteins | Peptides |
|---|---|---|
| Mouse | 142 | 197 |
| Human | 256 | 445 |

### Example 8

### Peptide Profiling is Reproducible

Because peptide profiling relies on the use of many data points to assess the degree of relatedness of many different samples, it is critical that the method be reproducible. This is confirmed on the samples described here. One such example, involving the peptide profile of yeast whole cell lysate, is shown here (Table 11, Table 12).

TABLE 11
Peptides observed for two repeat samples.

| | Total | Shared |
|---|---|---|
| Sample 1 | 776 | 686 |
| Sample 2 | 723 | 686 |

TABLE 12
Proteins observed for two repeat samples.

| | Total | Shared |
|---|---|---|
| Sample 1 | 304 | 259 |
| Sample 2 | 288 | 259 |

This analysis establishes the reproducibility of the process.

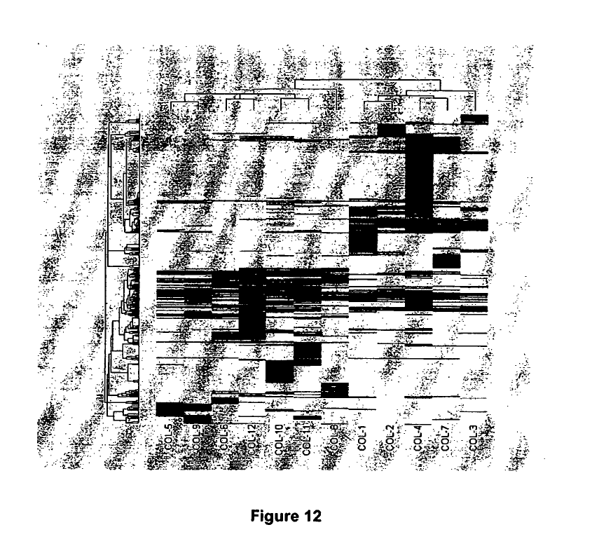

FIG. 12 is a representation of a reference database of protein profiles, incorporating both the identity, relative quantities, and overlap of peptides or proteins in various samples.

It will be appreciated that the description above relates to the preferred embodiments by way of example only. Many variations on the computer system and methods for delivering the invention will be obvious to those knowledgeable in the field, and such obvious variations are within the scope of the invention as described and claimed, whether or not expressly described.

All references, including journal articles, patents and patent applications, in this application are incorporated by reference herein in their entirety.

References

Beardsley, R. L., Karty, J. A. & Reilly, J. P. Enhancing the intensities of lysine-terminated tryptic peptide ions in matrix-assisted laser desorption/ionization mass spectrometry. Rapid Comm. Mass Spectrom.14, 2147-2153 (2000).

Eng, J. K., McCormack, A. L. & Yates, J. R. I. An approach to correlate tandem mass spectral data of peptides with amino acid sequences in a protein database. J. Am. Soc. Mass Spectrom. 6, 976-989 (1994).

Gygi, S. P., Rist, B., Gerber, S. A., Turecek, F., Geib, M. H. & Aebersold, R. Quantitative analysis of complex protein mixtures using isotope-coded affinity tags, Nat. Biotechnol. 17, 994-999 (1999).

Hale, J. E., Butler, J. P, Knierman, M. D. & Becker, G. W. Increased sensitivity of tryptic peptide detection by MALDI-TOF mass spectrometry is achieved by conversion of lysine to homoarginine. Anal. Biochem. 287, 110-117 (2000).

Kimmel, J. R., Guanidination of proteins. Meth. Enzymol. 11, 584-589 (1967).

Link, A. J., Eng, J., Schieltz, D. M., Carmack, E., Mize, G. J., Morris, D. R., Garvick, B. M. & Yates, J. R. Direct analysis of protein complexes using mass spectrometry. Nature Biotechnol. 17, 676-682 (1999).

Mann, M. & Wilm, M. Error-tolerant identification of peptides in sequence databases by peptide sequence tags. Anal. Chem. 66, 4390-4399 (1994).

Oda, Y., Huang, K., Cross, F. R., Cowburn, D. & Chait, B. T. Accurate quantitation of protein expression and site-specific phosphorylation. Proc. Natl. Acad. Sci. USA 96, 6591-6596 (1999).

Pandey, A. & Mann, M. Proteomics to study genes and genomes. Nature 405, 837-846 (2000).

1. A method for identifying the constituent proteins for a cell type, tissue or pathological sample using a database comprising peptide profile libraries wherein the libraries have multiple peptide sequences, comprising:
a) deriving a plurality of peptides from the cell type, tissue or pathological sample;
b) identifying the peptide species by liquid phase tandem mass spectroscopy sequencing;
c) compiling a data set or peptide profile containing the collection of peptide sequences obtained thereby; and
d) cross-tabulating with a collection of peptide sequences in the database.

2. The method of claim 1, wherein the step of deriving a plurality of peptides from the cell type, tissue or pathological sample further comprises the step of:
a) obtaining a peptide-containing extract of the cell type, tissue or pathological sample;
b) digesting the extract producing peptides with an enzyme, the enzyme capable of localizing mobile protons to the N-terminal amine and the side chains of the carboxy-terminal arginine or lysine residues;
c) separating the peptides by high pressure liquid chromatography apparatus;

3. The method of claim 2, wherein the enzyme comprises one selected from the group consisting of trypsin and endoproteinase LysC.

4. The method of claim 2, wherein the step of digesting the extract producing peptides further comprises the steps of:
a) dividing the extract into two equal portions;
b) derivatizing one of the two equal portions with a reagent, the reagent comprising one selected from the group consisting of o-methylisourea, homoarginine, canavanine, hydrazine, phenylhydrazine, and butyric acid derivatives.
c) combining the two portions.

5-7. Canceled.

8. A method for quantitating the relative abundance of proteins in two samples of a cell type, tissue or pathological sample using a database comprising peptide profile libraries wherein the libraries have multiple peptide sequences, comprising:
a) deriving a plurality of peptides from each sample of the cell type, tissue or pathological sample;
b) identifying the peptide species by tandem mass spectroscopy sequencing; compiling a data set or peptide profile containing the collection of peptide sequences obtained thereby;
c) cross-tabulating with a collection of peptide sequences in the database of peptide sequences; and
d) determining the relative abundance of the peptides and/or proteins.

9. Canceled.

10. The method of claim 8, wherein the step of deriving a plurality of peptides in two samples further comprises the step of:
a) obtaining a peptide-containing extract of each sample;
b) digesting separately the extracts producing peptides with an enzyme, the enzyme capable of localizing mobile protons to the N-terminal amine and the side chains of the carboxy-terminal arginine or lysine residues;
c) combining the two extracts; and
d) separating the peptides by high pressure liquid chromatography.

11. The method of claim 10, wherein the enzyme comprises one selected from the group consisting of trypsin and endoproteinase LysC.

12. The method of claim 8, wherein the step of digesting the extracts further comprises the step of derivatizing completely one of the two extracts with a reagent, the reagent comprising one selected from the group consisting of o-methylisourea, homoarginine, canavanine, hydrazine, phenylhydrazine, and butyric acid derivatives.

13-19. Canceled.

20. A method of comparing quantitative peptide profiles using a database of a plurality of peptide profile libraries, the method comprising:
a) receiving a selection of two or more of the peptide profile libraries;
b) determining the peptide profiles common to the selected peptide profile libraries and identifying profiles unique to each of selected peptide profile library; and
c) displaying the results of the determination.

21. The method of claim 20, wherein the correlation of a peptide profile against selected peptide profile libraries is determined by
P_{x,y}=[1n_{(j=1 to n)}Σ(Xⱼ−μₓ)(Yⱼ−μ_{y})]/[∂ₓ·∂_{y}]
where peptides common to two profiles score ‘1’ and peptides not shared between profiles score ‘0’.

22. The method of claim 21, wherein the peptides profiles are of cell fractions, the cell fractions comprising high molecular weight proteins, soluble proteins, membrane proteins, modified proteins, phosphoproteins, peptides terminating in lysine or arginine or the specific products of proteolytic enzymes or chemical derivatives of those products, peptides containing rare amino acids, and proteins isolated by binding to disease-specific affinity reagents.

23-24. Canceled.

25. The method of claim 22, wherein the rare amino acids comprise tryptophan and cysteine and amino acids comprising 5% or less of the amino acid representation.

26. The method of claim 22, wherein the disease-specific affinity reagents comprise polyclonal antibodies, toxin or drugs.

27. The method of claim 20, wherein the peptide profiles are of peptide sequences, the peptide sequences comprising mammalian peptide sequences.

28. The method of claim 20, wherein the peptide profiles are of peptide sequences, the peptide sequences comprising microbial peptide sequences.

29-30. Canceled.

31. The method of claim 20, wherein the step of receiving a selection of two or more of the peptide profile libraries for comparison comprises receiving an electronically transmitted file containing sequence and quantitative data.

32. The method of claim 20, wherein the results of the determination comprise a unique identifier for related peptide profiles.

33-34. Canceled.

35. The method of claim 20, further comprising the step of displaying the peptide profiles common to the selected peptide profile libraries.

36. The method of claim 20, further comprising the step of displaying the peptide profiles unique to the selected peptide profile libraries.

37. Canceled.
